(19) United States
(12) Patent Application Publication
(10) Pub. No.: US 2021/0393750 A1
(43) Pub. Date: Dec. 23, 2021

(54) INTER-ALPHA INHIBITOR PROTEINS AND METHODS OF USE THEREOF

(71) Applicant: ProThera Biologics, Inc., Providence, RI (US)

(72) Inventors: Yow-Pin Lim, Providence, RI (US); Denice Spero, Providence, RI (US); Andre Richard Santoso, Providence, RI (US)

(21) Appl. No.: 17/287,952

(22) PCT Filed: Oct. 24, 2019
(86) PCT No.: PCT/US19/57911

§ 371 Date: Apr. 22, 2021

Related U.S. Application Data
(60) Provisional application No. 62/750,019, filed on Oct. 24, 2018.

(51) Int. Cl. A61K 38/57; A61K 45/06; A61K 9/00; A61P 31/12; A61P 11/00; A61P 1/00; A61P 37/06; G01N 33/68; C07K 14/81; A23L 33/17; A23L 33/19; A23L 2/66; A23C 9/152; A23L 33/00

(52) U.S. Cl. CPC A61K 38/57; A23V 2002/00; A61K 9/0056; A61P 31/12; A61P 11/00; A61P 1/00; A61P 37/06; G01N 33/68; C07K 14/81; A23L 33/17; A23L 33/19; A23L 2/66; A23C 9/1526; A23L 33/40; G01N 2800/52; G01N 2333/811; A61K 45/06

(57) ABSTRACT

Featured are a foodstuff containing an inter-alpha inhibitor protein (lαlp) (e.g., lαl, Pαl, a heavy chain (e.g., H1, H2, H3, H4, and/or H5), a light chain (e.g., bikunin), or a combination thereof), methods of preparing a food-stuff containing an lαlp, and methods of treating and/or reducing the likelihood of developing a disease or condition (e.g., a disease or condition characterized by inflammation and/or low levels of an lαlp) in a subject in need thereof by administering a composition or a foodstuff containing an lαlp. Also featured are methods of purifying an lλlp (e.g., lαl, Pαl, a heavy chain (e.g., H1, H2, H3, H4, and/or H5), a light chain (e.g., bikunin), or a combination thereof) from milk.

### BACKGROUND

Inter-alpha inhibitor proteins (lαlps ) are a family of structurally related proteins found in plasma that are involved in inflammatory regulation and wound healing. The major forms of lαlps are Inter-alpha Inhibitor (lαl), which consists of two heavy chains (H1 & H2) and a single light chain (e.g., bikunin), and Pre-alpha Inhibitor (Pαl ) consisting of one heavy (H3) and one light chain (e.g., bikunin). lαlps are reduced during inflammatory processes such as sepsis and stroke. Prior studies indicate that lαlp levels are inversely correlated with morbidity and mortality in severe inflammatory conditions and that patients whose lαlp levels had recovered over time exhibited improved outcomes. Also, replenishing with exogenous lαlp in experimental models of severe inflammation provided recovery in multiple animal studies across species and indication areas.

There exists a need for improved compositions and methods for treating diseases and conditions characterized by inflammation and/or low lαlps levels in blood. In particular, there exists a need for developing compositions suitable for administration to infants (e.g., premature infants) for the treatment of inflammatory diseases.

### SUMMARY OF THE INVENTION

Featured is a foodstuff containing an inter-alpha inhibitor protein (lαlp) (e.g., lαl, Pαl, a heavy chain (e.g., H1, H2, H3, H4, and/or H5), a light chain (e.g., bikunin), or a combination thereof). The foodstuff containing an lαlp can be used to treat and/or reduce the likelihood of developing a disease or condition in a subject in need thereof by administering the foodstuff to the subject. The invention also features methods of purifying an lαlp (e.g., lαl, Pαl, a heavy chain (e.g., H1, H2, H3, H4, and/or H5), a light chain (e.g., bikunin), or a combination thereof) from milk (e.g., milk from a mammal, such as a human or domesticated ungulate). Additionally, featured are methods of determining whether a subject having a disease or condition is likely to respond to treatment with a foodstuff containing an lαlp, kits including a foodstuff containing an lαlp, and methods of treating or reducing the likelihood of developing necrotizing enterocolitis in a subject (e.g., an infant or newborn) by administering a composition, such as a foodstuff, containing an lαlp.

In a first aspect, the invention features a foodstuff containing an inter-alpha inhibitor protein (lαlp), in which the lαlp is present in the foodstuff in an amount of at least about 0.01 milligram per mg of the foodstuff.

In some embodiments, the lαlp is lαl, Pαl, H1, H2, H3, H4, H5, bikunin, or a combination thereof. In some embodiments, the lαlp includes lαl, Pαl, and/or bikunin. In some embodiments, the lαlp includes H1, H2, H3, H4, and/or H5. In some embodiments, the lαlp includes bikunin.

In some embodiments, the lαlp admixed with the foodstuff ranges in purity from about 85% to about 100% pure.

In some embodiments, the lαlp is present in the foodstuff in an amount of about 0.1 milligram (mg) to about 10 mg per mg of the foodstuff.

In some embodiments, the lαlp is present in the foodstuff in an amount of about 10 mg to about 1000 mg per liter (L) of the foodstuff.

In some embodiments, the lαlp is isolated from blood or milk. In some embodiments, the blood or milk is from a mammal. In some embodiments, the mammal is a human. In some embodiments, the mammal is a domesticated ungulate. In some embodiments, the domesticated ungulate is selected from the group consisting of a cow, goat, sheep, buffalo, camel, donkey, horse, reindeer, and yak. In some embodiments, the lαlp is expressed recombinantly in the mammal (e.g., the mammal is a transgenic mammal that is engineered to express lαlp). In some embodiments, the lαlp is a human lαlp.

In some embodiments, the lαlp has a biological activity. In some embodiments, the biological activity is selected from the group including a cytokine inhibitor activity, increase of cytokine activity, chemokine inhibitor activity, protease inhibitor activity, chondroitin sulfate binding, glycosaminoglygan binding activity, hyaluronic acid binding activity, complement binding activity, histone binding activity, Arg-Gly-Asp (RGD) domain binding activity, coagulation factor binding activity, cellular repair activity, and extracellular matrix protein binding activity. In some embodiments, the lαlp has a high trypsin inhibitory specific activity. In some embodiments, the trypsin inhibitory specific activity is between about 1000 IU/mg to about 2000 IU/mg.

In some embodiments, the foodstuff is pasteurized at a dry heat between about 50° C. and about 120° C.

In some embodiments, the foodstuff contains at least one pharmaceutically acceptable excipient, diluent, carrier, and/or stabilizer. In some embodiments, the stabilizer is selected from the group including albumin, polyethylene glycol, alpha-trehalose, amino acids, salts, glycerol, omega-amino acids, sugar, and combination thereof.

In some embodiments, the foodstuff is selected from the group including a beverage, a milk-based product, a baked good, a fruit and/or vegetable-based product, a grain and/or cereal-based product, a non-dairy product, an infant formula, an electrolyte product, a sports drink, a protein-based product, a nutritional supplement, a food additive, a flavoring, a sweetener, a preservative, a food coloring agent, and a fiber. In some embodiments, the milk-based product is selected from the group including milk, cream, butter, yogurt, kefir, ice cream, gelato, sherbet, custard, pudding, nougat, cheese, a whey product, and a casein product. In some embodiments, the baked good is selected from the group including a biscuit, bread, brownie, cake, casserole, cookie, cracker, pastry, pie, pizza, and tart. In some embodiments, the fruit and/or vegetable-based product is selected from the group including an oil, jelly, jam, marmalade, preserve, butter, puree, infant food, sauce, soup, and broth. In some embodiments, the non-dairy product is selected from the group including a cheese substitute, non-dairy yogurt, non-dairy cream, non-dairy butter, non-dairy ice cream, non-dairy milk, tofu, soy-based product, nut-based product, coconut-based product, and gelatin. In some embodiments, the cereal-based product is selected from the group including bread, pasta, oatmeal, breakfast cereal, tortilla, and grits. In some embodiments, the infant formula is selected from the group including a protein hydrolysate formula, metabolic formula, amino acid based formula, exempt infant formula, specialized formula, follow-on formula, and a toddler formula. In some embodiments, the electrolyte product is selected from the group including a pre-mixed solution, a dissolvable tablet, an edible gel, a concentrated solution, and a powder. In some embodiments, the electrolyte product and/or the sports drink is selected from the group consisting of an isotonic, hypertonic, and hypotonic solution. In some embodiments, the nutritional supplement and/or protein-based product is selected from the group consisting of a meal replacement product, protein or nutritional shake, protein bar, vitamin, energy drink, and prescribed foodstuff.

In some embodiments, the foodstuff is a solid. In some embodiments, the foodstuff is a liquid.

In some embodiments, the foodstuff further includes at least one additional therapeutic agent. In some embodiments, the additional therapeutic agent is selected from the group including an anti-cancer agent, an anti-inflammatory agent, an antiviral agent, an antibiotic agent, an antifungal agent, an antiparasitic agent, a bronchodilator, a vasopressor, a sedative, a complement inhibitor, an anti-coagulant, an immunomodulatory agent, an agent that induces tissue repair, an anticholinergic, an antidiarrheal, an antidepressant, a prokinetic agent, a laxative, a neurotransmitter, an antispasmodic, and a pain reliever.

In some embodiments, the foodstuff of the foregoing aspect or any of the foregoing embodiments is for the treatment of a disease or condition in a subject in need thereof. In some embodiments, the disease or condition is associated with a low level of lαlp in a subject as compared to a reference level of lαlp. In some embodiments, the disease or condition is associated with an altered level of at least one cytokine and/or chemokine in a subject as compared to a reference level of the at least one cytokine and/or chemokine. In some embodiments, the cytokine and/or chemokine is TNF-α.

In another aspect, the invention features a method of treating, reducing the symptoms of, inhibiting progression of, or reducing the likelihood of developing a disease or condition in a subject by administering to the subject a foodstuff containing a therapeutically effective amount of an lαlp. In some embodiments, the foodstuff is a foodstuff of the foregoing aspect or of any of the foregoing embodiments.

In some embodiments, the method further includes the step of determining the level of an lαlp in the subject. In some embodiments, the level of the lαlp in the subject is determined prior to administration. In some embodiments, the level of the lαlp in the subject is determined after administration.

In some embodiments, the disease or condition is associated with a low level of lαlp in the subject as compared to a reference level of lαlp.

In another aspect, the invention features a method of determining whether a subject having a disease or condition is likely to respond to treatment with a foodstuff containing lαlp including the steps of: (a) optionally determining a pre-treatment level of one or more lαlps in the subject; (b) administering a therapeutically effective amount of the foodstuff of any of the foregoing aspects or embodiments to the subject; and (c) determining the level of one or more of the lαlp in the subject after an initial treatment period, in which an increase in the level of at least one of the lαlp in the subject indicates that the subject is likely to respond favorably to treatment with the foodstuff. In some embodiments, the method further includes the step of monitoring the level of one or more lαlp-related biomarkers in the subject prior to and/or post administration of the foodstuff comprising the lαlp.

In another aspect, the invention features a method of determining whether a subject having a disease or condition is likely to respond to treatment with a foodstuff containing an lαlp including the steps of: (a) optionally determining a pre-treatment level of one or more lαlp-related biomarkers in the subject; (b) administering a therapeutically effective amount of the foodstuff of any of the foregoing aspects or embodiments to the subject; and (c) determining the level of one or more of the lαlp-related biomarkers in the subject after an initial treatment period, in which a change in the level of at least one of the lαlp-related biomarkers in the subject indicates that the subject is likely to respond favorably to treatment with the foodstuff.

In another aspect, the invention features a method of optimizing therapeutic efficacy of a treatment of a subject having a disease or condition with a foodstuff containing an lαlp, the method including the steps of: (a) optionally determining a pre-treatment level of one or more lαlps in the subject; (b) administering a therapeutically effective amount of the foodstuff of any of the foregoing aspects or embodiments to the subject; (c) determining the level of one or more of the lαlps in the subject after an initial treatment period, in which (i) an increase in the level of at least one of the lαlps in the subject indicates that the foodstuff can be administered to the subject at a similar or reduced dosage or frequency, and (ii) a decrease or plateau in the level of at least one of the lαlps in the subject indicates that the foodstuff can be administered to the subject at an increased frequency or dosage; and (d) optionally adjusting the frequency and/or the dosage at which the foodstuff is administered to the subject.

In some embodiments of any of the foregoing aspects, the disease or condition is associated with an elevated level of at least one cytokine and/or chemokine in the subject as compared to a reference level of the at least one cytokine and/or chemokine. In some embodiments, the cytokine and/or chemokine is selected from the group including IL-1⊕, TNF-α, INF-α, IL-6, IL-10, INF-γ, and IL-8. In some embodiments, administration of the foodstuff results in a decrease in or down-regulation of one or more of the cytokines and/or chemokines.

In some embodiments of any of the foregoing aspects, the disease or condition is selected from the group including acute inflammatory disease, acute and chronic neurological and neurodegenerative disorders, sepsis, severe shock, septic shock, organ transplantation, organ failure, surgery, autoimmune disease, rheumatoid arthritis, multiple sclerosis, lupus, cancer, cancer metastasis, metabolic disorders, cachexia, trauma and/or injury, tissue damage, exposure to a toxin, liver disease, infectious disease, lung and respiratory disease, heart disease, kidney disease, ischemia, gastrointestinal disease, necrotizing enterocolitis, systemic inflammatory response syndrome (SIRS), rhinitis, exposure to a toxin, meningitis, acute pancreatitis, preeclampsia, preterm labor, primary immunodeficiency syndrome, and acquired immunodeficiency syndrome (AIDS). In some embodiments, the inflammatory disease is an inflammatory bowel disease. In some embodiments, the inflammatory bowel disease is Crohn's disease. In some embodiments, the lung disease is an acute lung injury. In some embodiments, the acute lung injury is acute respiratory distress syndrome (ARDS). In some embodiments, the acute lung injury is pneumonia. In some embodiments, the trauma and/or injury is a wound. In some embodiments, the ischemia is ischemia/reperfusion injury. In some embodiments, the ischemia is hypoxic ischemia. In some embodiments, the ischemia is hypoxic ischemic encephalopathy. In some embodiments, the disease or condition is necrotizing enterocolitis. In some embodiments, the tissue damage is internal scarring, tissue damage resulting from organ transplantation or surgery, tissue damage resulting from inflammation, disease, or injury, lung tissue damage (e.g., lung tissue damage caused by asthma, chronic obstructive pulmonary disease (COPD), bronchitis, cystic fibrosis, pneumonia, emphysema, ARDS, pneumoconiosis, lung cancer, interstitial lung disease, pulmonary fibrosis, or sarcoidosis), brain tissue damage (e.g., brain tissue damage caused by ischemia, hypoxia, epilepsy, TBI, hypoxic ischemic encephalopathy, or stroke), gastrointestinal tissue damage (e.g., gastrointestinal tissue damage caused by an autoimmune or inflammatory disease or condition (e.g., an inflammatory bowel disease, such as Crohn's disease or ulcerative colitis) or intestinal ischemia), or vascular tissue damage (e.g., vascular tissue damage caused by inflammation or injury).

In some embodiments of any of the foregoing aspects, administration of the foodstuff reduces the frequency and/or occurrence of at least one symptom of the disease or condition in the subject, relative to an untreated subject. In some embodiments, the symptom is selected from the group including organ failure; hypoxemia; bilateral lung opacities; respiratory failure; dizziness, lightheadedness and/or fainting; fatigue; shortness of breath and/or labored breathing; cough; fever; abnormal vital signs, such as increased heart rate; low blood pressure; rapid breathing, chest pain and/or pressure; heart palpitations; edema; swelling, pain, and/or bloating of the abdomen; discoloration of the abdomen; pain in the lower joints and/or rectum; bloody stool; bowel obstruction; nausea; flatulence; loss of appetite; weight loss and/or poor weight gain; slow growth; diarrhea; poor feeding; vomiting; bleeding; redness, swelling, pain, tenderness and/or heat of the tissues proximal to a wound; blueish coloring of nails and/or lips; and the need for mechanical ventilation.

In some embodiments of any of the foregoing aspects, the foodstuff is administered at a dosage of about 1 mg/kg body weight to about 5 g/kg body weight of the subject. In some embodiments, the lαlp is present in the foodstuff in an amount of about 0.1 milligram (mg) to about 10 mg per mg of the foodstuff. In some embodiments, the lαlp is present in the foodstuff in an amount of about 10 mg to about 1000 mg per liter (L) of the foodstuff.

In some embodiments of any of the foregoing aspects, the foodstuff is administered over a treatment period of at least 1 day.

In some embodiments of any of the foregoing aspects, the method further includes administering an additional therapeutic agent.

In another aspect, the invention features a method of purifying an lαlp including the steps of: (a) separating a fraction of milk comprising the lαlp, and (b) purifying the lαlp from the fraction of milk, in which the lαlp has a purity ranging from about 85% to about 100%. In some embodiments, the separating and/or purifying includes a clarification step, a chromatography step, a precipitation step, and/or a solid phase extraction step. In some embodiments, the chromatography step includes anion-exchange and/or affinity chromatography. In some embodiments, the precipitation step includes contacting the fraction of milk with an agent that produces a precipitate lacking the lαlp.

In some embodiments, the method further includes exposing the lαlp to a pH of about 5.5 or lower (e.g., a pH of about 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.1, 3.0, 2.9, 2.8, 2.7, 2.6, 2.5. 2.4, 2.3, 2.2, 2.1, or about 2.0), optionally a pH of about 4.2 to about 5.2.

In some embodiments, fat and/or milk proteins are removed from the sample prior to chromatographic separation.

In some embodiments, the milk is from a mammal. In some embodiments, the mammal is a human. In some embodiments, the mammal is a domesticated ungulate. In some embodiments, the domesticated ungulate is selected from the group including a cow, goat, sheep, buffalo, camel, donkey, horse, reindeer, and yak. In some embodiments, the lαlp is expressed recombinantly in the mammal and secreted into the milk of the mammal (e.g., the mammal is a transgenic mammal that is engineered to express and secrete lαlp). In some embodiments, the lαlp is a human lαlp.

In another aspect, the invention features a method of purifying an lαlp including the steps of: (a) providing a mammal containing a milk-producing cell transfected with a transgene that includes: (i) a nucleic acid sequence encoding the lαlp, (ii) a milk-specific promoter, said promoter being operably linked to the nucleic acid sequence encoding the lαlp, and (iii) a leader sequence encoding a protein secretory signal that enables secretion of the lαlp by the milk-producing cell; and (b) purifying the lαlp from milk collected from the mammal. In some embodiments, the lαlp is exogenous to the mammal. In some embodiments, the mammal is a domesticated ungulate. In some embodiments, the domesticated ungulate is selected from the group consisting of a cow, goat, sheep, buffalo, camel, donkey, horse, reindeer, and yak. In some embodiments, the lαlp is a human lαlp.

In another aspect, the invention features a method of making a composition for oral consumption by admixing an lαlp with a foodstuff.

In some embodiments, the amount of lαlp admixed with the foodstuff ranges from about 0.1 milligram (mg) to about 10 mg per mg of the foodstuff.

In some embodiments, the amount of lαlp admixed with the foodstuff ranges from about 10 mg to about 1000 mg per liter (L) of the foodstuff.

In some embodiments, the lαlp is isolated from blood or milk. In some embodiments, the blood or milk is from a mammal. In some embodiments, the mammal is a human. In some embodiments, the mammal is a domesticated ungulate. In some embodiments, the domesticated ungulate is selected from the group including a cow, goat, sheep, buffalo, camel, donkey, horse, reindeer, and yak. In some embodiments, the lαlp is expressed recombinantly in the mammal (e.g., the mammal is a transgenic mammal that is engineered to express lαlp). In some embodiments, the lαlp is a human lαlp.

In some embodiments of any of the foregoing aspects, the lαlp has an apparent molecular weight of between about 60 kDa to about 280 kDa. In some embodiments, the molecular weight is determined by sodium dodecyl sulfate polyacrylamide gel electrophoresis.

In some embodiments of any of the foregoing aspects, the lαlp has an in vivo half-life of greater than one hour. In some embodiments, the in vivo half-life is greater than five hours.

In some embodiments of any of the foregoing aspects, the lαlp has a biological activity. In some embodiments, the biological activity is selected from the group including a cytokine inhibitor activity, increase of cytokine activity, chemokine inhibitor activity, protease inhibitor activity, chondroitin sulfate binding, glycosaminoglygan binding activity, hyaluronic acid binding activity, complement binding activity, histone binding activity, Arg-Gly-Asp (RGD) domain binding activity, coagulation factor binding activity, cellular repair activity, and extracellular matrix protein binding.

In some embodiments of any of the foregoing aspects, the lαlp has a high trypsin inhibitory specific activity. In some embodiments, the trypsin inhibitory specific activity is between about 1000 IU/mg to about 2000 IU/mg.

In some embodiments of any of the foregoing aspects, the foodstuff is selected from the group including a beverage, a milk-based product, a baked good, a fruit and/or vegetable-based product, a grain and/or cereal-based product, a non-dairy product, an infant formula, an electrolyte product, a sports drink, a protein-based product, a nutritional supplement, a food additive, a flavoring, a sweetener, a preservative, a food coloring agent, and a fiber. In some embodiments of any of the foregoing aspects, the milk-based product is selected from the group including milk, cream, butter, yogurt, kefir, ice cream, gelato, sherbet, custard, pudding, nougat, cheese, a whey product, and a casein product. In some embodiments of any of the foregoing aspects, the baked good is selected from the group including a biscuit, bread, brownie, cake, casserole, cookie, cracker, pastry, pie, pizza, and tart. In some embodiments of any of the foregoing aspects, the fruit and/or vegetable-based product is selected from the group including an oil, jelly, jam, marmalade, preserve, butter, puree, infant food, sauce, soup, and broth. In some embodiments of any of the foregoing aspects, the non-dairy product is selected from the group including a cheese substitute, non-dairy yogurt, non-dairy cream, non-dairy butter, non-dairy ice cream, non-dairy milk, tofu, soy-based product, nut-based product, coconut-based product, and gelatin. In some embodiments of any of the foregoing aspects, the cereal-based product is selected from the group including bread, pasta, oatmeal, breakfast cereal, tortilla, and grits. In some embodiments of any of the foregoing aspects, the infant formula is selected from the group including a protein hydrolysate formula, metabolic formula, amino acid based formula, exempt infant formula, specialized formula, follow-on formula, and a toddler formula. In some embodiments of any of the foregoing aspects, the electrolyte product is selected from the group including a pre-mixed solution, a dissolvable tablet, an edible gel, a concentrated solution, and a powder. In some embodiments of any of the foregoing aspects, the electrolyte product and/or the sports drink is selected from the group including an isotonic, hypertonic, and hypotonic solution. In some embodiments of any of the foregoing aspects, the nutritional supplement and/or protein-based product is selected from the group including a meal replacement product, protein or nutritional shake, protein bar, vitamin, energy drink, and prescribed foodstuff.

In some embodiments of any of the foregoing aspects, the foodstuff is a solid.

In some embodiments of any of the foregoing aspects, the foodstuff is a liquid.

In some embodiments of any of the foregoing aspects, the lαlp is present in the foodstuff in an amount of about 0.1 mg to about 10 mg per mg of the foodstuff.

In some embodiments of any of the foregoing aspects, the lαlp is present in the foodstuff in an amount of about 10 mg to about 1000 mg per L of the foodstuff.

In some embodiments of any of the foregoing aspects, the lαlp is about 1% to about 60% of the volume of the foodstuff.

In some embodiments of any of the foregoing aspects, the method further includes admixing at least one additional therapeutic agent with the foodstuff.

In another aspect, the invention features a kit containing the foodstuff of any of the foregoing aspects or embodiments and instructions for therapeutic use.

In another aspect, the invention features a kit containing a composition including an lαlp, a foodstuff, instructions for admixing the composition with the foodstuff, and, optionally, instructions for therapeutic use.

In some embodiments of any of the foregoing aspects, the composition further includes an additional therapeutic agent.

In another aspect, the invention features a method of treating, reducing the symptoms of, inhibiting progression of, or reducing the likelihood of developing necrotizing enterocolitis in a subject in need thereof by administering to the subject a composition containing in admixture a therapeutically effective amount of an lαlp.

In some embodiments of any of the foregoing aspects, the lαlp is lαl, Pαl, H1, H2, H3, H4, H5, bikunin, or a combination thereof. In some embodiments of any of the foregoing aspects, the lαlp includes lαl, Pαl, and/or bikunin. In some embodiments of any of the foregoing aspects, the lαlp includes H1, H2, H3, H4, and/or H5. In some embodiments of any of the foregoing aspects, the lαlp includes bikunin.

In some embodiments of any of the foregoing aspects, the lαlp ranges in purity from about 85% to about 100% pure.

In some embodiments of any of the foregoing aspects, the lαlp is isolated from blood or milk. In some embodiments of any of the foregoing aspects, the blood or milk is from a mammal. In some embodiments, the mammal is a human.

In some embodiments of any of the foregoing aspects, the method includes the step of administering the foodstuff of any one of the foregoing aspects or embodiments.

In some embodiments of any of the foregoing aspects, the lαlp is administered about every 4 to about 120 hours.

In some embodiments of any of the foregoing aspects, the lαlp is administered at least once a day. In some embodiments, the lαlp is administered at least twice a day.

In some embodiments of any of the foregoing aspects, the lαlp is administered over a treatment period.

In some embodiments of any of the foregoing aspects, the treatment period is about 1 day to about 14 days. In some embodiments of any of the foregoing aspects, the treatment period is about 1 week to about 3 weeks. In some embodiments of any of the foregoing aspects, the treatment period is about 1 week to about 4 weeks. In some embodiments of any of the foregoing aspects, the treatment period of is about 1 month to about 12 months. In some embodiments of any of the foregoing aspects, the treatment period is at least 1 year.

In some embodiments of any of the foregoing aspects, the method further includes determining the level of an lαlp and/or an lαlp-related biomarker in the subject.

In some embodiments of any of the foregoing aspects, the lαlp-related biomarker is selected from the group including histone, extracellular histone, histone/Pαl complexes, histone/lαl complexes, histone lαl/Pαl complexes, TNF-α, IL-6, IL-10, IL-1, IL-1ra, IL1B, IL-8, MCP-1, MIP-2, C-reactive protein (CRP), procalcitonin (PCT), cytokine-induced neutrophil chemoattractant/KC, UTI, complement components C1, C2, C3, C4, C5, C6, C7, C8, C9, membrane attack complex, Factor B, Factor D, MASP-1, and MASP-2, or fragments thereof. In some embodiments, the level of the lαlp and/or an lαlp-related biomarker in the subject is determined prior to administration of the composition. In some embodiments, the level of the lαlp and/or lαlp-related biomarker in the subject is determined after administration of the composition.

In some embodiments of any of the foregoing aspects, the lαlp is administered at a dosage of about 1 mg/kg body weight to about 5 g/kg body weight.

In some embodiments of any of the foregoing aspects, the composition contains a pharmaceutically acceptable excipient, diluent, or carrier. In some embodiments, the composition is a solid. In some embodiments, said solid is a tablet, capsule, or suppository. In some embodiments, the composition is a liquid. In some embodiments, the composition is formulated for injection, infusion, inhalation, insufflation, or nebulization, or for oral, rectal, or topical administration. In some embodiments, the injection is intravenous, intraperitoneal, or intracerebral injection. In some embodiments, the infusion is fetal infusion.

In some embodiments of any of the foregoing aspects, the method further includes administering an additional therapeutic agent.

In some embodiments of any of the foregoing aspects, the additional therapeutic agent is selected from the group including an anti-cancer agent, an anti-inflammatory agent, an antiviral agent, an antibiotic agent, an antifungal agent, an antiparasitic agent, a bronchodilator, a vasopressor, a sedative, a complement inhibitor, an anti-coagulant, an immunomodulatory agent, an agent that induces tissue repair, an anticholinergic, an antidiarrheal, an antidepressant, a prokinetic agent, a laxative, a neurotransmitter, an antispasmodic, and a pain reliever.

In some embodiments of any of the foregoing aspects, the subject is a mammal. In some embodiments, the subject is a human. In some embodiments, the subject is a fetus, neonate, infant, child, adolescent, or adult. In some embodiments, the infant is a premature infant.

In some embodiments of any of the foregoing aspects, the method includes administering a therapeutically effective amount of the foodstuff of any of the foregoing aspects of embodiments to the subject.

In some embodiments of any of the foregoing aspects, the lαlp is a human lαlp.

### Definitions

As used herein, the singular form “a,” “an,” and “the” includes plural references unless indicated otherwise.

As used herein, the term “about” means +/−10% of the recited value.

As used herein, the term “acute respiratory distress syndrome” or “ARDS” refers to an acute form of lung injury characterized by widespread inflammation of the lungs that may include, for example, diffuse alveolar injury, surfactant dysfunction, an innate immune response, and/or abnormal coagulation. ARDS is also typically characterized by bilateral pulmonary infiltrates and severe hypoxemia in the absence of evidence for cardiogenic pulmonary edema. The severity of hypoxemia necessary to make the diagnosis of ARDS can be defined by the ratio of the partial pressure of oxygen in the patient's arterial blood (PaO₂) to the fraction of oxygen in the inspired air (FiO₂) (PaO₂/FiO₂). A definition of ARDS depends on the relationship of the timing of the onset of clinical symptoms to the lung injury, radiographic changes, origin of edema, and severity of the symptoms based on the measurement of PaO₂/FiO₂ ratio on 5 cm of H₂O continuous positive airway pressure (CPAP). The 2012 Berlin definition for ARDS classified ARDS into three categories based on the degree of hypoxemia as determined by PaO₂/FiO₂: mild ARDS (PaO₂/FiO₂200-300 mm Hg), moderate ARDS (PaO₂/FiO₂ 100-200 mm Hg), and severe ARDS (PaO₂/FiO₂ 100 mm Hg) (see, e.g., The ARDS Definition Task Force, JAMA 307(23):2526-2533, 2012). The American-European Consensus Conference on ARDS (AECC) classified ARDS in terms of a PaO₂/FiO₂ ratio of less than 200 mm Hg, whereas acute lung injury (ALI), which is less severe than ARDS, was characterized by a PaO₂/FiO₂ of less than 300 mm Hg (Bernard et al., Am. J. Respir. Crit. Care Med. 143(3 Pt 1):818-824, 1994). It is to be understood that the term “ARDS” encompasses any suitable clinical definition for ARDS known in the art, including the 2012 Berlin definition or the 1994 AECC definition.

As used herein, the term “acute respiratory failure” refers to a condition in which fluid builds up in the air sacs of the lung, thereby reducing the release of oxygen into the blood stream (hypoxemia) and removal of CO₂ (hypercapnia) and leading to a hypoxic condition in the subject. The hypoxic condition can reduce oxygen delivery to organs, which can result in organ failure. Failure to remove CO₂ from blood can result in respiratory acidosis characterized by an increase in blood pH.

As used herein, “administering” is meant a method of giving a dosage of a substance (e.g., an lαlp) or a composition (e.g., an lαlp-containing composition, such as an lαlp-containing foodstuff of the invention) to a subject. The lαlps utilized in the methods described herein can be administered, for example, orally, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, topically, locally, by inhalation, by injection, by infusion, by continuous infusion, by localized perfusion bathing target cells directly, by catheter, by lavage, in creams, or in lipid compositions. The method of administration can vary depending on various factors (e.g., the substance or composition being administered and the severity of the condition, disease, or disorder being treated). In some embodiments, an lαlp-containing composition, such as an lαlp-containing foodstuff of the invention, is administered to a subject orally.

The terms “biomarker” and “lαlp-related biomarker” as used herein refer to a substance, e.g., a protein, nucleic acid, or chemical agent, which can be detected in a sample, for example, a bodily fluid, such as blood, that is indicative of, e.g., a disease or disorder that is caused by, related to, or associated with the level of an lαlp in a subject. In some embodiments, the lαlp-related biomarker is a protein that forms a complex with an lαlp, e.g., by direct and/or indirect binding to the lαlp. Non-limiting examples of lαlp-related biomarkers include histone, extracellular histone, histone/Pαl complexes, histone/Ial complexes, histone lαl/Pαl complexes, TNF-α, IL-6, IL-10, IL-1, IL-1ra, IL1B, IL-8, MCP-1, MIP-2, C-reactive protein (CRP), procalcitonin (PCT), cytokine-induced neutrophil chemoattractant/KC, UTI, complement components C1, C2, C3, C4, C5, C6, C7, C8, C9, membrane attack complex, Factor B, Factor D, MASP-1, and MASP-2, and/or fragments thereof. The level of an lαlp-related biomarker, may serve, e.g., as an indicator of a particular subtype or symptom of a disease or disorder (e.g., a disease or disorder associated with a low level of an lαlp and/or inflammation) characterized by certain, molecular, pathological, histological, and/or clinical features. lαlp-related biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers. The level of a lαlp-related biomarker may be determined by methods known in the art, e.g., by conventional protein detection assays including, without limitation, ELISA-based assays, immunoblot assays (e.g., Western blot assays), mass spectrometry (such as, for instance, matrix-assisted laser desorption ionization-time of flight (MALDI-TOF) mass spectrometry and electrospray ionization (ESI) mass spectrometry), and spectroscopic methods, such as nuclear magnetic resonance (NMR), infrared (IR) spectroscopy, and UV-Vis spectroscopy, among others. Additional methods, e.g., for determining the abundance of RNA transcripts in a sample can be performed using established techniques known in the art, including quantitative, reverse-transcription polymerase chain reaction (qRT-PCR) assays, and RNA sequencing assays (RNA-Seq), among others.

As used herein, the term “change” refers to an alteration (e.g., an increase or decrease) in the level of a substance, such as an lαlp (e.g., lαl, Pαl, a heavy chain (e.g., H1, H2, H3, H4, and/or H5), a light chain (e.g., bikunin), or a combination thereof) and/or an lαlp-related biomarker (e.g., histone, extracellular histone, histone/Pαl complexes, histone/lαl complexes, histone lαl/Pαl complexes, TNF-α, IL-6, IL-10, IL-1, IL-1ra, IL1B, IL-8, MCP-1, MIP-2, C-reactive protein (CRP), procalcitonin (PCT), cytokine-induced neutrophil chemoattractant/KC, UTI, complement components C1, C2, C3, C4, C5, C6, C7, C8, C9, membrane attack complex, Factor B, Factor D, MASP-1, and MASP-2, or fragments thereof) as detected by standard methods known in the art. As used herein, a change includes at least about a 1% (e.g., 10%, 20%, 30%, 40%, 50%, 60%, 70%, 80%, or 90%) or more alteration (e.g., an increase or decrease) in the level of the substance assayed (e.g., an lαlp protein and/or an lαlp-related biomarker) in a sample obtained from a subject (e.g., a human) relative to, e.g., the level of the substance in a healthy subject.

As used herein, the term “complement activation” refers to the activation of complement components that react with one another to induce a series of inflammatory responses that help to fight infection. The complement system activates through a triggered-enzyme cascade.

As used herein, the term “complement components” refers to complement system proteins in the classical pathway, lectin pathway, and the alternate complement pathways, including but not limited to C1, C2, C3 (e.g., C3a and C3b), C4 (e.g., C4b), C5 (e.g., C5a and C5b), C6, C7, C8, C9, membrane attack complex, Factor B, Factor D, mannan-binding lectin associated serine protease 1 (MASP-1), and MASP-2, and fragments thereof.

In this disclosure, the terms “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

A “disorder” is a condition that would benefit from treatment including, but not limited to, chronic and acute disorders or diseases including those pathological conditions which predispose the subject to the disorder in question.

As used herein, “inhibiting progression” of a disorder or disease means to delay, defer, hinder, slow, retard, stabilize, and/or postpone development of the disease or disorder, as described herein. This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, the term “inter-alpha inhibitor proteins” or “lαlps” refers to large, multi-component glycoproteins in a family of structurally related serine protease inhibitors. lαlps have been shown to be important in the inhibition of an array of proteases including neutrophil elastase, plasmin, trypsin, chymotrypsin, Granzyme K, preprotein convertase, furin, cathepsin G, and acrosin. In human plasma, lαlps are found at relatively high concentrations (400-800 mg/L). Unlike other inhibitor molecules, this family of inhibitors typically includes a combination of polypeptide chains (light and heavy chains) covalently linked by a chondroitin sulfate chain. The heavy chains of lαlps (H1, H2, and H3) are also called hyaluronic acid (HA) binding proteins. The major forms of lαlps found in human plasma are inter-alpha-inhibitor (lαl), which contains two heavy chains (H1 and H2) and a single light chain (L), and pre-alpha-inhibitor (Pαl), which contains one heavy (H3) and one light chain (L). Another lαlp is the light chain (also termed bikunin (bi-kunitz inhibitor) with two Kunitz domains), which is known to broadly inhibit plasma serine proteases. Another lαlp is the heavy chain-related molecule H4, which circulates in the blood without linkage to bikunin. Yet another lαlp is the heavy chain-related molecule H5. lαl and Pαl present in the plasma fraction have an apparent molecular weight of between about 60 kDa to about 280 kDa.

As used herein, the term “pneumonia” refers to an inflammatory condition in the lung which is the result of an infection caused by bacteria, viruses, fungi, or other microorganisms such as parasites (e.g., protozoan parasites). Pneumonia is typically diagnosed with chest X-rays, by clinical assessments, sputum culture, and/or blood culture. In a patient having pneumonia, the air sacs fill with fluid (e.g., pus) and may become solid. The infection and related inflammation may affect both lungs, one lung, or only certain lobes of a lung. The term “pneumonia” encompasses any suitable clinical definition or classification of pneumonia known in the art, for example, the CRB-65 criteria, CURB-65 criteria (see, e.g., Lim et al., Thorax 58(5):377-382, 2003) or the pneumonia severity index (PSI) (see, e.g., Fine et al., N. Engl. J. Med. 336(4):243-250, 1997). These criteria are also described in Wente et al. Respiratory Medicine 109:157-169, 2015, which is incorporated herein by reference in its entirety. The term pneumonia encompasses any suitable type of pneumonia, including but not limited to hospital-acquired pneumonia (HAP), health care-associated pneumonia (HCAP), nursing home-acquired pneumonia (NHAP), ventilator-associated pneumonia (VAP), and community acquired pneumonia (CAP), including severe CAP (sCAP).

As used herein, the terms “prevent,” “preventing,” “prevention,” “prophylactic treatment” and the like refer to reducing the probability of developing a disorder or condition in a subject, who does not have, but is at risk of or is susceptible to developing, a disease, disorder, or condition.

As used herein, the phrase “reducing the likelihood of developing” refers to prophylactic treatment of a patient susceptible to, or otherwise at risk of, a particular disease, syndrome, or condition or is at risk of a current disease, syndrome, or condition increasing in its degree of severity

As used here, the term “reference” is meant a standard or a control condition. For example, a sample, cell, or tissue that is used as a reference is one that may be obtained, e.g., from a healthy subject or from a subject prior to treatment, and used for comparison with an unknown sample, cell, or tissue, respectively. A reference level may be the level of an lαlp and/or lαlp-related biomarker.

As used herein, the term “respiratory failure” refers to a condition resulting from inadequate gas exchange by the respiratory system in which insufficient oxygen passes from the lungs into blood and CO₂ is expelled.

As used herein, the term “sepsis” refers to a systemic response to an infection (referred to herein as “infectious sepsis”) or to a non-infectious process associated with acute tissue injury and innate immune activation (referred to interchangeably herein as “sterile inflammation” or “sterile sepsis”), which can lead to tissue damage, organ failure, and death. Infectious sepsis can result from an infection caused by bacteria, viruses, fungi, or other microorganisms such as parasites (e.g., protozoan parasites). Sterile sepsis can occur after hemorrhagic shock, polytrauma, pancreatitis, transplant rejection, autoimmune disease, or ischemia/reperfusion and is not associated with the presence of a known infection.

As used herein, the term “subject” refers to a mammal, including, but not limited to, a human or non-human mammal, such as a primate, bovine, equine, porcine, ovine, feline, or canine. The subject may be a patient.

As used herein, the term “treating” refers to reducing or ameliorating a disorder and/or symptoms associated therewith. It will be appreciated that, although not precluded, treating a disorder or condition does not require that the disorder or symptoms associated therewith be completely eliminated.

### DESCRIPTION OF THE DRAWINGS

FIG. 1 is a series of images showing the starting material, flow-through, wash fractions, and eluted fraction obtained during the chromatographic isolation of lαlp from human and bovine milk. The starting material, flow-through, wash fractions, and eluate were separated on a 7.5% SDS-PAGE gel (FIG. 1, top), and transferred onto a nitrocellulose membrane for Western blot analysis (FIG. 1, bottom). lαlp was detected using a biotinylated rabbit polyclonal antibody against rat lαlp (R22C) that cross-reacts with human and bovine lαlp. Both the 125 kDa and 250 kDa bands of lαlp, corresponding to Pαl and lαl, respectively, were detected in the elution fraction of human and bovine milk (arrows in FIG. 1, bottom, Lanes 5 and 10). The lanes of the SDS-PAGE gel and Western blot are as follows: Lane 1: Starting material—bovine milk; Lane 2: Flow-through—bovine milk; Lane 3: Wash fraction 1 (300 mM NaCl)—bovine milk; Lane 4: Wash fraction 2 (pH 4.5)—bovine milk; Lane 5: Elution—bovine milk; Lane 6: Starting material—human milk; Lane 7: Flow-through—human milk; Lane 8: Wash fraction 1 (300 mM NaCl)—human milk; Lane 9: Wash fraction 2 (pH 4.5)—human milk; Lane 10: Elution—human milk.

### DETAILED DESCRIPTION OF THE INVENTION

The invention features a foodstuff containing (e.g., in admixture) an inter-alpha inhibitor protein (lαlp) (e.g., lαl, Pαl, a heavy chain (e.g., H1, H2, H3, H4, and/or H5), a light chain (e.g., bikunin), or a combination thereof) and methods of treating and/or reducing the likelihood of developing a disease or condition in a subject in need thereof by administering the foodstuff to the subject. The invention also features methods of purifying an lαlp (e.g., lαl, Pαl, a heavy chain (e.g., H1, H2, H3, H4, and/or H5), a light chain (e.g., bikunin), or a combination thereof) from milk. Additionally, the invention features methods of treating or reducing the likelihood of developing necrotizing enterocolitis in a subject (e.g., an infant or newborn) by administering a composition, such as a foodstuff, containing an lαlp (e.g., lαl, Pαl, a heavy chain (e.g., H1, H2, H3, H4, and/or H5)).

Compositions and Methods of Making a Foodstuff Containing an lαlp

A foodstuff for oral consumption can be prepared by admixing an inter-alpha inhibitor protein (lαlp) (e.g., lαl, Pαl, a heavy chain (e.g., H1, H2, H3, H4, and/or H5), a light chain (e.g., bikunin), or a combination thereof) with a food or beverage (e.g., a commercially prepared, premade, prepackaged, convenience, ready-to-eat, portion-controlled, single-serve, and/or homemade food or beverage). The foodstuff can include an amount of an lαlp known in the art to be therapeutic (see, e.g., U.S. Pat. No. 7,932,365, International Patent Application Publication No. WO2009154695, and U.S. Patent Application Publication No. 2009/0190194, each of which is incorporated herein by reference in its entirety) and described herein. Prior to admixing with a foodstuff, the lαlp (e.g., lαl, Pαl, a heavy chain (e.g., H1, H2, H3, H4, and/or H5), a light chain (e.g., bikunin), or a combination thereof) may be in the form of a solid (e.g., a freeze-dry protein powder, such as is produced, e.g., by lyophilization) or a liquid (e.g., a composition of lαlp in a food safe excipient, diluent, carrier, and/or stabilizer).

The foodstuff may be one that is readily available, e.g., from a grocery store or pharmacy, or one that is readily prepared (e.g., by following a recipe). An lαlp (e.g., lαl, Pαl, a heavy chain (e.g., H1, H2, H3, H4, and/or H5), a light chain (e.g., bikunin), or a combination thereof) may be admixed with a foodstuff during the process of preparing the foodstuff, e.g., by following a recipe that includes the step of combining an amount of an lαlp (e.g., an amount of an lαlp in the range of at least about 0.01 milligram or more (e.g., 0.01, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, or more milligrams) per mg of the foodstuff, e.g., about 0.1 milligram (mg) to about 10 mg per mg of the foodstuff, or in the range of about 10 mg to about 3000 mg per liter (L) of the foodstuff) with the ingredient(s) of the foodstuff. An lαlp may also be admixed with a foodstuff after the foodstuff has been otherwise prepared (e.g., by following a recipe) and is ready for human consumption. For example, an amount of an lαlp (e.g., an amount of an lαlp in the range of about 10 mg to about 3000 mg per liter (L) of the foodstuff) can be admixed into a beverage (e.g., water, milk, coffee, tea, or any other fluid safe for human consumption), e.g., just prior to consumption.

Non-limiting examples of a foodstuff (e.g., a solid and/or a liquid foodstuff) suitable for admixing with an lαlp (e.g., lαl, Pαl, a heavy chain (e.g., H1, H2, H3, H4, and/or H5), a light chain (e.g., bikunin), or a combination thereof) include water, milk-based products, baked goods, fruit and/or vegetable-based products, grain and/or cereal-based products, non-dairy products, infant formulas, electrolyte products, sports drinks, protein-based products, and nutritional supplements.

A milk-based product that can be prepared to contain an lαlp may be selected from the group including milk (e.g., milk of any percentage, fortified milk, raw milk, pasteurized milk, human breast milk (including, e.g., colostrum), milk from any animal including, e.g., domesticated ungulates), dry or powdered milk, cream, butter, yogurt, kefir, ice cream, gelato, sherbet, custard, pudding, nougat, cheese, whey and/or casein products (e.g., protein shakes, powders, or bars). The composition of milk differs among species. Human milk contains about 1% protein, about 4% fat, about 7% sugar (e.g., lactose), and supplies about 72 kcal of energy per 100 grams, while cow milk contains about 3% protein, 3% fat, and 5% sugar (e.g., lactose), about 1% minerals (e.g., calcium, magnesium, potassium, and sodium), and supplies about 66 kcal of energy per 100 grams. The invention contemplates the preparation of a foodstuff including in admixture an lαlp having a protein, fat, sugar, and mineral composition comparable to that of milk (e.g., milk from a mammal, such as a human or domesticated ungulate, e.g., a cow, goat, sheep, buffalo, camel, donkey, horse, reindeer, and yak).

A baked good that can be prepared to contain an lαlp may be selected from the group including a biscuit, bread, brownie, cake, casserole, cookie, cracker, pastry, pie, pizza, and tart.

A fruit and/or vegetable-based product that can be prepared to contain an lαlp may be selected from the group including oil, jelly, jam, marmalade, preserve, butter, fruit and/or vegetable purees, infant food, sauce, soup, and broth.

A grain and/or cereal-based product that can be prepared to contain an lαlp may be any foodstuff made from wheat, rice, oats, cornmeal, barley, or another cereal grain, and may be selected from the group including bread, pasta, oatmeal, breakfast cereal, tortilla, and grits.

A non-dairy product that can be prepared to contain an lαlp may be selected from the group including a cheese substitute, non-dairy yogurt, non-dairy cream, non-dairy butter, non-dairy ice cream, non-dairy milk, tofu, soy-based product, nut-based product, coconut-based product, and gelatin. An infant formula that can be prepared to contain in admixture an lαlp may be any formula that is based on milk (e.g., milk from a mammal, such as a human or domesticated ungulate, e.g., a cow, goat, sheep, buffalo, camel, donkey, horse, reindeer, and yak; milk from an edible bean, such as a soybean; milk from a nut, such as an almond, walnut, hazelnut, or cashew; or milk from a coconut), a protein hydrolysate formula, a metabolic formula, an amino acid based formula, an exempt infant formula, a specialized formula for premature infants, a follow-on formula, or a toddler formula. An exempt infant formula is an infant formula intended for commercial or charitable distribution that is represented and labeled for use by infants who have inborn errors of metabolism or low birth weight, or who otherwise have unusual medical or dietary problems. Non-limiting examples of exempt infant formula that can be admixed with an lαlp (e.g., lαl, Pαl, a heavy chain (e.g., H1, H2, H3, H4, and/or H5), a light chain (e.g., bikunin), or a combination thereof) to make a foodstuff of the invention include, e.g., metabolic formulas produced by ABBOTT NUTRITION® (e.g., Cyclinex-1, Glutarex-1, Hominex-1, I-Valex-1, Ketonex-1, Phenex-1, Propimex-1, and Tyrex-1), MEAD JOHNSON NUTRITIONALS® (e.g., Phenyl Free 1, BCAD 1, GA, HCY 1, LMD, OA 1, TYROS 1, and WND 1), and SHS International Limited (e.g., MSUD Anamix Early Years, IVA Anamix Early Years GA1 Anamix Early Years, HCU Anamix Early Years, MMA/PA Anamix Early Years, Periflex Early Years, Tyr Anamix Early Years, and SOD Anamix Early Years); formulas for premature infants produced by ABBOTT NUTRITION® (e.g., SIMILAC® Special Care 20 Cal w/Iron, SIMILAC® Special Care 24 Cal w/Iron, SIMILAC® Special Care 24 Cal High Protein, SIMILAC® Special Care 30 Cal w/Iron, and SIMILAC EXPERT CARE® NeoSure), MEAD JOHNSON NUTRITIONALS® (e.g., ENFAMIL® Premature Low Iron 20 Calorie, ENFAMIL® Premature w/Iron 20 Calorie, ENFAMIL® Premature Low Iron 24 Calorie, ENFAMIL® Premature w/Iron 24 Calorie, ENFAMIL® EnfaCare, ENFAMIL® Premature High Protein 24 Calorie, and ENFAMIL® Premature 30 Calorie), Nestle Infant Nutrition (e.g., Gerber Good Start Nourish, Gerber Good Start Premature 20, Gerber Good Start Premature 24 High Protein, Gerber Good Start Premature 24, and Gerber Good Start Premature 30), and PBM Nutritionals (e.g., a 22 cal/oz milk-based infant formula with DHA and ARA for conditions such as prematurity); extensively hydrolyzed whey protein isolate formulas such as Gerber Extensive HA produced by Nestle Infant Nutrition; protein hydrolysate formulas produced by ABBOTT NUTRITION® (e.g., SIMILAC EXPERT CARE® Alimentum), MEAD JOHNSON NUTRITIONALS® (e.g., NUTRAMIGEN®, PREGESTIMIL® 20 Calorie, PREGESTIMIL® 24 Calorie, and NUTRAMIGEN® with Enflora LGG); amino acid-based formulas produced by ABBOTT NUTRITION® (e.g., ELECARE® with DHA and ARA), MEAD JOHNSON NUTRITIONALS® (e.g., PURAMINO®), Nestle Infant Nutrition (e.g., ALFAMINO®), and SHS International Limited (e.g., Neocate Infant w/DHA and ARA); and miscellaneous exempt infant formulas produced by ABBOTT NUTRITION® (e.g., Calcilo XD, Liquid Protein Fortifier, PRO-PHREE®, PROVIMIN®, RCF No Added Carbohydrate Soy Infant Formula Base, SIMILAC EXPERT CARE® for Diarrhea, SIMILAC® Human Milk Fortifier, SIMILAC® Extensively Hydrolyzed Protein Human Milk Fortifier Concentrated Liquid, SIMILAC® Human Milk Fortifier Concentrated Liquid, and SIMILAC® PM 60/40), MEAD JOHNSON NUTRITIONALS® (e.g., Product 3232A, ENFAMIL® Human Milk Fortifier Acidified Liquid, ENFAMIL® Human Milk Fortifier Powder, and ENFAPORT®), and PROLACTA BIOSCIENCES®, Inc. (e.g., Prolact Plus Human Milk Fortifiers (+4, +6, +8, and +10), Prolact CR Human Milk Caloric Fortifier, Prolact RTF 24 Human Milk-Based Premature Infant Formula, Prolact RTF 26 Human Milk-Based Premature Infant Formula, and Prolact RTF 28 Human Milk-Based Premature Infant Formula).

Sports drinks and electrolyte products that can be prepared to contain an lαlp include, but are not limited to, pre-mixed solutions, dissolvable tablets, edible gels, concentrated solutions, and powders. Sports drinks suitable for use in the methods and compositions of the invention may be selected from the group consisting of an isotonic sports drink, a hypertonic sports drink, and a hypotonic sports drink. Examples, of popular sports drinks include, 100PLUS®, 10-K THIRST QUENCHER®, ACCELERADE®, ALL SPORT®, AQUARIUS®, coconut water, GATORADE®, HERBALIFE H30 PRO®, ISOSTAR®, LUCOZADE SPORT®, MONSTER®, MUSCLE MILK®, POCARI SWEAT®, POWERADE®, REVIVE®, SQWINCHER®, STAMINADE®, and VEMMA THIRST®.

The electrolyte products that can be prepared to contain an lαlp include, but are not limited to isotonic, hypertonic, and hypotonic solutions (e.g., PEDIALYTE®). Generally, the electrolyte product is lower in sugar as compared to most sports drinks (e.g., there are ˜100 calories/liter in PEDIALYTE® as compared to ˜200 calories/liter in GATORADE®). Additionally, the electrolyte product generally has a higher oral rehydration salts (ORS) concentration, for example, sodium (e.g., ˜1,035 mg/L in PEDIALYTE® compared to ˜465 mg/L in GATORADE)) and potassium (e.g., ˜780 mg/L in PEDIALYTE® compared to ˜127 mg/L in GATORADE)) concentration as compared to most sports drinks. Additionally, sucrose is not generally used in electrolyte products (e.g., PEDIALYTE®) because it is associated with the risk of worsening the symptoms of diarrhea by drawing water into the intestine and increasing the risk of dehydration. Flavored electrolyte products may include synthetic sweeteners (e.g., sucralose and acesulfame potassium). For example, PEDIALYTE® includes the following ingredients: water, dextrose, less than 2% of citric acid, natural and artificial flavor, potassium citrate, salt, sodium citrate, sucralose, acesulfame potassium, zinc gluconate, and Yellow 6.

ORS solutions that can be prepared to contain an lαlp are available as either pre-prepared fluids or packets of ORS ready for mixing with a fluid, such as water. ORS solutions are effective in patients with dehydration regardless of age, cause, or type of electrolyte imbalance (e.g., hyponatremia, hypernatremia, or isonatremia) as long as their kidneys are functioning adequately. ORS solutions are generally prepared to contain about 2% glucose and about 50 to about 90 mEq/L of sodium (Na). Sports drinks, sodas, juices, and similar drinks should not be used for rehydration, as they generally have an insufficient amount of Na and too much carbohydrate (e.g., glucose) to take advantage of Na/glucose cotransport in the gut, which is optimized for a Na:glucose ratio of about 1:1. However, the osmotic effect of excess carbohydrate may contribute to additional fluid loss. An exemplary ORS solution includes salt (e.g., about 0.1-4 grams NaCl, such as about 2.6 grams NaCl), trisodium citrate dihydrate (e.g., about 1-4 grams C₆H₅Na₃O₇, 2H₂O, such as about 2.9 grams C₆H₅Na₃O₇, 2H₂O), potassium chloride (e.g., about 0.1-3 grams KCl, such as about 1.5 grams KCl), and glucose (e.g., about 10-20 grams C₆H₁₂O₆, such as about 13.5 grams C₆H₁₂O₆) per liter of fluid. An ORS solution containing between about 75 mmol sodium/ to about 90 mmol sodium,/ may contain an amount of sodium that is too high for, e.g., severely malnourished children, Thus, ORS solutions prepared with an lαlp for use in malnourished children, e.g., due to dehydration caused by diarrhea, can be prepared with less than about 45 mmol sodium/L and about 40 mmol potassium/L.

For tablets for oral use, carriers that are commonly used include lactose and com starch. Lubricating agents, such as magnesium stearate, may also be added. For oral administration in a capsule form, useful diluents include lactose and dried corn starch. When aqueous suspensions and/or emulsions are administered orally, the lαlp may be suspended or dissolved in an oily phase combined with emulsifying and/or suspending agents. If desired, certain sweetening and/or flavoring and/or coloring agents may be added.

A nutritional supplement or protein-based product that can be prepared to contain an lαlp may be selected from the group including a meal replacement product, a protein or nutritional shake (e.g.,

ENSURE)), a protein bar, a vitamin, an energy drink, a prescribed foodstuff product. A nutritional supplement (e.g., ENSURE)), for example, may include the following ingredients, water, corn maltodextrin, sugar, milk protein concentrate, soy oil, soy protein isolate sucromalt, canola oil; less than 0.5% of corn oil, magnesium phosphate, potassium citrate, cellulose, gel, natural and artificial flavor, salt, calcium phosphate, sodium citrate, calcium carbonate, potassium chloride, choline chloride, ascorbic acid, cellulose, gum, monoglycerides, soy lecithin, carrageenan, potassium hydroxide, liquid sucralose, ferrous sulfate, dl-alpha-tocopheryl acetate, acesulfame potassium zinc sulfate, niacinamide manganese sulfate, calcium pantothenate, cupric sulfate, vitamin a palmitate, thiamine chloride hydrochloride pyridoxine hydrochloride, riboflavin, folic acid chromium chloride biotin sodium molybdate, potassium iodide, sodium selenate, phylloquinone, vitamin D3, and cyanocobalamin.

Additionally, an lαlp may be admixed with a food additive, flavoring, sweetener (e.g., sugar, sugar-alternative, honey, agave nectar), preservative (e.g., antimicrobial additives, such as benzoic acid, sodium benzoate, hydroxybenzoate and derivatives thereof, lactic acid, nitrite, nitrate, propionic acid, sodium propionate, sulfur dioxide, sulfites, sorbic acid, and sodium sorbate; antioxidants, such as ascorbic acid, sodium ascorbate, butylated hydroxytouluene, butylated hydroxyanisole, gallic acid, sodium gallate, sulfur dioxide, sulfites, and tocopherols; and natural preservatives, such as rosemary extract, hops, salt, sugar, vinegar, alcohol, diatomaceous earth, and castor oil), nutritional supplement (e.g., a vitamin), food coloring agent, or a fiber (e.g., a fiber powder). Beverages, such as carbonated beverages (e.g., sodas and seltzers), teas, coffee, herbal teas and tinctures, tonic water, water, and can also be used to prepare an lαlp-containing foodstuff.

The lαlp may be added to a foodstuff in an amount in the range of at least about 0.01 milligram or more (e.g., 0.01, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, or more milligrams) per mg of the foodstuff, e.g., about 0.1 milligram (mg) to about 10 mg per mg of the foodstuff (e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, or 100 mg per mg of foodstuff) or in the range of about 10 mg to about 3000 mg per liter (L) of the foodstuff (e.g., 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 22300, 2400, 2500, 2600, 2700, 2800, 2900, or 3000 mg per liter (L) of foodstuff). The lαlp may be added to a foodstuff at a percentage of about 1% to about 80% (e.g., 1, 2, 3, 4, 5, 6, 7, 8, 9, 10, 15, 20, 25, 30, 35, 40, 45, 50, 55, 60, 65, 70, 75, or 80%) of the total weight (mg) or volume (L) of the foodstuff. The lαlps may be admixed with the foodstuff in a variety of combinations. For example, any one or more of the lαlps (e.g., lαl, Pαl, H1, H2, H3, H4, H5, and bikunin) may be admixed into a foodstuff individually or in a combination, such as lαl and Pαl; lαl and/or bikunin; Pαl and/or bikunin; or lαl, Pαl, and/or bikunin with H1, H2, H3, H4, and/or H5. The lαlps (e.g., lαl and/or Pαl) may be present in the foodstuff in a physiological proportion. Physiological proportions may be, for example, the proportions found in a person or animal that is healthy and/or the ratio of lαl and Pαl that appears naturally in human plasma. Physiological proportions are typically from between about 60% to about 80% lαl (e.g., about 60, 61, 62, 63, 64, 65, 66, 67, 68, 69, 70, 71, 72, 73, 74, 75, 76, 77, 78, 79, or about 80% lαl) and between about 20% to about 40% Pαl (e.g., 20, 21, 22, 23, 24, 25, 26, 27, 28, 29, 30, 31, 32, 33, 34, 35, 36, 37, 38, 39, or about 40% Pal). However, it is to be understood that physiological proportions may vary from these ranges, for example, due to normal variation in genetic makeup of subjects.

An lαlp (e.g., lαl, Pαl, a heavy chain (e.g., H1, H2, H3, H4, and/or H5), a light chain (e.g., bikunin), or a combination thereof) admixed with a foodstuff may be substantially free from other proteins and biological components that are found with the lαlp in its natural source (e.g., blood or milk). For example, an lαlp purified from blood may be substantially free of a detectable amount of, e.g., albumins, IVIg, globulins (e.g., alpha2-macroglobulin, gamma globulins, beta-2 microglobulin, and haptoglobin), fibrinogen, prothrombin, clotting factors, alpha-1-antitrypsin, alpha-1-acid glycoprotein, alpha-1-fetoprotein, ceruloplasmin, complement component 3, complement component 4, c-reactive protein (CRP), lipoproteins (e.g., chylomicrons, very Low-density lipoprotein (VLDL), low-density lipoprotein (LDL), high-density lipoproteins (HDL)), transferrin, prothrombin, and mannose-binding protein (MBP). For example, an lαlp purified from milk may be substantially free of a detectable amount of, e.g., casein, lactalbumin (whey), and lactose. Any suitable materials and methods can be used to isolate and purify the lαlp, e.g., methods described herein, and methods described in in International Publication No. WO2005046587 and in Provisional Application Nos. 62/490,003 and 62/614,333, herein incorporated in their entirety, to obtain an lαlp from blood and/or milk ranging in purity from about 85% to about 100% pure (e.g., 85, 86, 87, 88, 89, 90, 91, 92, 93, 94, 95, 96, 97, 98, 99, or 100% pure). The milk and/or blood can be obtained from any mammal, such as a human or domesticated ungulate (e.g., a cow, goat, sheep, buffalo, camel, donkey, horse, reindeer, and yak). In particular instances, the lαlp may be a human lαlp expressed recombinantly in the mammal (e.g., the mammal is a transgenic mammal that is engineered to express lαlp). The lαlp has an apparent molecular weight of between about 60 kDa to about 280 kDa, which can be determined by any appropriate method known in the art, e.g., by sodium dodecyl sulfate polyacrylamide gel electrophoresis.

The biochemical and/or biophysical properties of an lαlp (e.g., lαl, Pαl, a heavy chain (e.g., H1, H2, H3, H4, and/or H5), a light chain (e.g., bikunin), or a combination thereof) may be assessed prior to or after being admixed with a foodstuff by any appropriate method known in the art. An lαlp (e.g., lαl, Pαl, a heavy chain (e.g., H1, H2, H3, H4, and/or H5), a light chain (e.g., bikunin), or a combination thereof) obtained from milk and/or blood that is useful for admixing with a foodstuff of the invention has an in vivo half-life of about one hour or greater (e.g., 1, 2, 3, 4, 5, 6, 7 8, 9, 10, 11, 12, 13, 14, 15, 16, 17, 18, 19, 20 hours). The lαlp also has a biological activity, such as an activity selected from the group consisting of a cytokine inhibitor activity, increase of cytokine activity, chemokine inhibitor activity, protease inhibitor activity (e.g., serine protease inhibitor activity), chondroitin sulfate binding, glycosaminoglygan binding activity, hyaluronic acid binding activity, complement binding activity, histone binding activity, Arg-Gly-Asp (RGD) domain binding activity, coagulation factor binding activity, cellular repair activity, and extracellular matrix protein binding activity. The lαlp also has a high trypsin inhibitory specific activity, e.g., between about 1000 IU/mg to about 2000 IU/mg (e.g., 1000, 1100, 1200, 1300, 1400, 1500, 1600, 1700, 1800, 1900, or 2000 IU/mg).

A foodstuff including an lαlp (e.g., lαl, Pαl, a heavy chain (e.g., H1, H2, H3, H4, and/or H5), a light chain (e.g., bikunin), or a combination thereof) may also be prepared by admixing at least one (e.g., 1, 2, 3, 4, 5, 6, 7, 8, 9, or 10 or more) additional therapeutic agent with the foodstuff. Examples of therapeutic agents include anti-cancer agents, anti-inflammatory agents, antiviral agents, antibiotic agents, antifungal agents, antiparasitic agents, bronchodilators, vasopressors, sedatives, complement inhibitors, anti-coagulants, immunomodulatory agents, agents that induce tissue repair, anticholinergics, antidiarrheals, antidepressants, prokinetic agents, laxatives, neurotransmitters, antispasmodics, and pain relievers.

### Therapeutic Methods

### Subject to be Treated

The invention features methods of treating a subject with a disease, condition, or symptoms thereof, e.g., characterized by inflammation and/or low levels of an lαlp. Methods of identifying a subject suitable for treatment with a composition (e.g., a foodstuff) containing an lαlp (e.g., lαl, Pαl, a heavy chain (e.g., H1, H2, H3, H4, and/or H5), a light chain (e.g., bikunin), or a combination thereof) are known in the art. Exemplary subjects suitable for treatment using the methods of the invention include the patient populations described in, e.g., International Patent Application Publication Nos. WO2014039987, WO2005046587, and WO2009154695, each of which is incorporated herein by reference in its entirety. The lαlp-containing compositions (e.g., foodstuffs) described herein can be used to treat a fetus, a neonate (e.g., a newborn less than four weeks old), an infant (e.g., a premature infant), a child, an adolescent, or an adult. Non-limiting examples of diseases and conditions suitable for treatment with an lαlp-containing composition (e.g., a foodstuff with admixed lαlp) include lung diseases (e.g., acute respiratory distress syndrome (ARDS), pneumonia, community-acquired pneumonia (CAP), chronic obstructive pulmonary disease (COPD)), acute and chronic neurological and neurodegenerative disorders (e.g., central nervous system (CNS) diseases (e.g., ischemia in the brain, hypoxic ischemic brain injury (e.g., neonatal), hypoxic ischemic encephalopathy, stroke (e.g., ischemic hemorrhagic stroke), Alzheimer's disease, Parkinson's disease, traumatic brain injury (TBI), neuropathic pain, and epilepsy)), sepsis, severe shock, septic shock, cancer (e.g., cancer metastasis), metabolic disorders (e.g., diabetes type I/II, cachexia), heart disease (e.g., myocardial infarction, and congestive heart failure), ischemia (e.g., ischemia/reperfusion injury), kidney disease (e.g., acute kidney injury and polycystic kidney disease, dialysis), trauma/major injury with blood loss (e.g., wound healing); tissue damage (e.g., tissue repair following tissue or organ transplantation or surgery, repair of tissue or organ damage resulting from inflammation, disease, or injury, tissue repair to reduce internal scarring, lung tissue repair (e.g., tissue repair in a subject having lung tissue damage caused by asthma, chronic obstructive pulmonary disease (COPD), bronchitis, cystic fibrosis, pneumonia, emphysema, ARDS, pneumoconiosis, lung cancer, interstitial lung disease, pulmonary fibrosis, or sarcoidosis), brain tissue repair (e.g., tissue repair in a subject having brain tissue damage caused by ischemia, hypoxia, epilepsy, TBI, hypoxic ischemic encephalopathy, or stroke), gastrointestinal tissue repair (e.g., tissue repair in a subject having gastrointestinal tissue damage caused by an autoimmune or inflammatory disease or condition (e.g., an inflammatory bowel disease, such as Crohn's disease or ulcerative colitis) or intestinal ischemia), vascular tissue repair (e.g., tissue repair in a subject having vascular tissue damage caused by inflammation or injury), muscle tissue repair, hepatic tissue repair, or cardiac tissue repair); for infection (e.g., bacterial, viral (e.g., by H1 N1, H5N1 (Avian Flu), Dengue, Zika, and others viral infections), parasitic, or fungal infections, including treatments for biodefense (e.g., against anthrax and other bioterror/emerging pathogens)); liver disease (e.g., chronic liver injury, fatty liver disease (Nonalcoholic steatohepatitis (NASH)), acute inflammatory disease (e.g., inflammatory bowel diseases, e.g., Crohn's disease), necrotizing enterocolitis (NEC), acute pancreatitis preeclampsia, preterm labor, organ transplantation and organ failure, surgery (e.g., pre- and post-surgery), autoimmune diseases (e.g., rheumatoid arthritis (RA), multiple sclerosis (MS), systemic lupus erythematosus, alopecia areata, autoimmune hemolytic anemia, autoimmune hepatitis, dermatomyositis, diabetes (type 1), juvenile idiopathic arthritis, glomerulonephritis, Graves' disease, Guillain-Barré syndrome, idiopathic thrombocytopenic purpura, myasthenia gravis, myocarditis, pemphigus/pemphigoid, pernicious anemia, polyarteritis nodosa, polymyositis, primary biliary cirrhosis, psoriasis, scleroderma/systemic sclerosis, Sjogren's syndrome, thyroiditis, uveitis, vitiligo, and granulomatosis with polyangiitis (GPA/Wegener's)), rhinitis, exposure to a toxin (e.g., anthrax related toxins (e.g., exotoxins, lethal toxin (LT), and edema toxin (ET)), meningitis, primary immunodeficiency syndrome, and acquired immunodeficiency syndrome (AIDS).

Treatment with a composition (e.g., a foodstuff) containing an lαlp (e.g., lαl, Pαl, a heavy chain (e.g., H1, H2, H3, H4, and/or H5), a light chain (e.g., bikunin), or a combination thereof) can completely or partially ameliorate and/or abolish some or all of the symptoms of a disease or condition described herein, decrease the severity of the symptoms, delay the onset of symptoms, or lessen the progression and/or severity of subsequently developed symptoms. In particular, administration of an lαlp (e.g., a composition containing an lαlp admixed with a foodstuff) to a subject in need thereof can reduce circulating levels of pro-inflammatory mediators (e.g., cytokines and chemokines), vascular cell adhesion protein 1 (VCAM1-), intracellular adhesion molecule 1 (ICAM-1), and lαlp-related biomarkers (e.g., histone, extracellular histone, histone/Pαl complexes, histone/Ial complexes, histone lαl/Pαl complexes, TNF-α, IL-6, IL-10, IL-1, IL-1ra, IL1B, IL-8, MCP-1, MIP-2, C-reactive protein (CRP), procalcitonin (PCT), cytokine-induced neutrophil chemoattractant/KC, UTI, complement components C1, C2, C3, C4, C5, C6, C7, C8, C9, membrane attack complex, Factor B, Factor D, MASP-1, and MASP-2, or fragments thereof), or can remove (e.g., decrease the level of) and/or inactivate (e.g., decrease the activity of) proteases (e.g., serine proteases, such as trypsin, elastase (e.g., human leukocyte elastase (HLE)), plasmin, cathepsin G, and granzyme K), resulting in improved survival, reduced morbidity, reduced severity and/or occurrence of symptoms, and increased time to treat an underlying disease or condition (e.g., by combination therapy). Administration of an lαlp (e.g., a composition containing an lαlp admixed with a foodstuff) to a subject in need thereof can also reduce the severity of inflammation, which can be assessed by measuring sedimentation rate (erythrocyte sedimentation rate). A reduction in the circulating level of a pro-inflammatory mediator, the level of VCAM-1, the level of ICAM-1, the level of an lαlp-related biomarker, the sedimentation rate, or the activity level of a protease may be measured, e.g., in comparison to a baseline level (e.g., a known level of the pro-inflammatory mediator, a known level of VCAM-1, a known level of ICAM-1, a known level of the lαlp-related biomarker, a known sedimentation rate, or a known activity level of the protease, respectively, that is associated with, e.g., a healthy subject). For example, a reduction in the level of at least one pro-inflammatory mediator, a reduction in the level of at least one lαlp-related biomarker, a reduction in the level of VCAM-1, a reduction in the level of ICAM-1, a reduction in the sedimentation rate, or a reduction in the activity level of at least one protease of about 5% (e.g., 5%, 10%, 15%, 20%, 25%, 30%, 35%, 40%, 45%, 50%, 55%, 60%, 65%, 70%, 75%, 80%, 85%, 90%, 95% or more) compared to a respective baseline level may result in improved survival, reduced morbidity, reduced severity and/or occurrence of symptoms, and increased time to treat an underlying disease or condition (e.g., by combination therapy). In some instances, the baseline level of a pro-inflammatory mediator, the baseline level of an lαlp-related biomarker, the baseline level of VCAM-1, the baseline level of ICAM-1, the baseline sedimentation rate, and/or the baseline level of activity of a protease may be obtained from a healthy, untreated subject (e.g., a subject untreated with an lαlp (e.g., lαl, Pαl, a heavy chain (e.g., H1, H2, H3, H4, and/or H5), a light chain (e.g., bikunin), or a combination thereof). In other instances, the baseline level of a pro-inflammatory mediator, the baseline level of an lαlp-related biomarker, the baseline level of VCAM-1, the baseline level of ICAM-1, the baseline sedimentation rate, and/or the baseline level of activity of a protease may be obtained from a subject having a disease or condition described herein prior to treatment (e.g., treatment with an lαlp (e.g., lαl, Pαl, a heavy chain (e.g., H1, H2, H3, H4, and/or H5), a light chain (e.g., bikunin), or a combination thereof)), e.g., for comparison to a sample from the subject after treatment.

### Administration

The lαlp-containing foodstuff may be administered to the subject one (e.g., 1, 2, 3, 4, 5, 6, 7, 8, 9, 10, 11, 12, 13, 14, 15, 16, 17, 18, 19, 20) or more times every 1, 2, 3, 4, 5, 6, 8, 12, 24, 48, 72, 96, or 120 hours; one or more times every 1, 2, 3, 4, 5, 6, 7, 8, 9, 10, 11, 12, 13, or 14 or more days; or one or more times every 1 week (e.g., one or more times every 2, 3, or 4 weeks). The lαlp-containing foodstuff may be administered over a treatment period of about 1 day to about 14 days, 1 week to about 4 weeks, 1 month to about 12 months, or about 1 year or more. In other cases, the lαlp-containing foodstuff is administered for an indeterminate period of time or a limited period of time, e.g., periodically or continuously to a subject (e.g., as a prophylactic). For example, the foodstuff could be administered to a subject at least 10, 15, 20, 30, 60, or 120 minutes before the onset or occurrence of a disease, condition, or symptom thereof. Additionally, a composition including an lαlp may be administered to a subject in need thereof upon diagnosis of a disease, condition, or symptom thereof or after development of a disease, condition, or symptom thereof. A subject may also be treated by administration of milk (e.g., human breast milk, including, e.g., colostrum) that naturally contains an lαlp or that has been fortified by addition of exogenous lαlp. For example, an adult subject may be administered cow or human milk as the therapeutic for the treatment of any of the diseases or conditions described herein.

### Therapeutic Regimen

A foodstuff containing lαlps (e.g., lαl, Pαl, a heavy chain (e.g., H1, H2, H3, H4, and/or H5), a light chain (e.g., bikunin), or a combination thereof) can be administered to a subject to treat one or more of the diseases and/or conditions described herein so as to provide a dosage of lαlp ranging from about 1 mg/kg to 50 mg/kg (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 mg/kg), such as a dosage of between about 10 mg/kg to about 30 mg/kg. The foodstuff can be administered one or more times every 1, 2, 3, 4, 5, 6, 7, 8, 9, 10, 11, 12, 24, 48, 72, 96, or 24 hours, every 1, 2, 3, 4, 5, 6, 7, 8, 9, 10, 11, 12, 13, or 14 or more days, or every 1, 2, 3, or 4 weeks or more, or as needed. Lower or higher doses of lαlp in the foodstuff than those recited above may be used. Specific dosage and treatment regimens for any particular subject may depend upon a variety of factors, including the activity of the specific composition (e.g., a foodstuff) employed, the age, body weight, general health status, sex, diet, time of administration, rate of excretion, drug combination, the severity and course of the disease (e.g., the patient's condition and/or symptoms), the subjects disposition to the disease, and the judgment of the treating medical professional (e.g., the physician).

Upon improvement of the patient's condition, a maintenance dose of an lαlp composition (e.g., a foodstuff) or combination therapy may be administered. Subsequently, the dosage or frequency of administration, or both, may be reduced, as a function of the reduction in symptoms, to a level at which the improved condition is maintained. When the symptoms have been alleviated to a desired level, treatment may cease. Subjects may, however, require intermittent treatment on a long-term basis upon any recurrence of disease symptoms. Improvement of the condition may also be judged based upon the level of an lαlp (e.g., lαl, Pαl, a heavy chain (e.g., H1, H2, H3, H4, and/or H5), a light chain (e.g., bikunin), or a combination thereof) in a biological sample derived from the patient (e.g., blood (e.g., whole blood, plasma, or serum), bronchial lavage fluid (BALF), sputum, urine, cerebrospinal fluid (CSF), or a tissue biopsy (e.g., a liver or intestinal biopsy). Improvement of the disease or condition may also be judged using assays to detect the severity of inflammation, such as measurements of sedimentation rate (erythrocyte sedimentation rate). Improvement of the disease and/or condition may also be judged based upon the level of pro-inflammatory mediators (e.g., cytokines and chemokines), VCAM-1, ICAM-1, and lαlp-related biomarkers (e.g., histone, extracellular histone, histone/Pαl complexes, histone/lαl complexes, histone lαl/Pαl complexes, TNF-α, IL-6, IL-10, IL-1, IL-1ra, IL1B, IL-8, MCP-1, MIP-2, C-reactive protein (CRP), procalcitonin (PCT), cytokine-induced neutrophil chemoattractant/KC, UTI, complement components C1, C2, C3, C4, C5, C6, C7, C8, C9, membrane attack complex, Factor B, Factor D, MASP-1, and MASP-2, or fragments thereof) determined, for example, by immunological methods. A reduction in the circulating level of a pro-inflammatory mediator, the level of VACM-1, the level of ICAM-1, the sedimentation rate, or the level of an lαlp-related biomarker may be measured, e.g., in comparison to a baseline level (e.g., a known level of the pro-inflammatory mediator, a known level of VACM-1, a known level of ICAM-1, a known sedimentation rate, or a known level of the lαlp-related biomarker, respectively, that is associated with, e.g., a healthy subject). For example, a reduction in the level of at least one pro-inflammatory mediator, a reduction in the level of VACM-1, a reduction in the level of ICAM-1, a reduction in the sedimentation rate, and/or a reduction in the level of at least one lαlp-related biomarker of about 5% (e.g., 5%, 10%, 15%, 20%, 25%, 30%, 35%, 40%, 45%, 50%, 55%, 60%, 65%, 70%, 75%, 80%, 85%, 90%, 95% or more) compared to a respective baseline level may indicate or result in improved survival, reduced morbidity, reduced severity and/or occurrence of symptoms, or increased time to treat an underlying disease or condition (e.g., by combination therapy). In some instances, the baseline level of a pro-inflammatory mediator, the baseline level of VACM-1, the baseline level of ICAM-1, the baseline sedimentation rate, and/or the baseline level of an lαlp-related biomarker may be obtained from a healthy, untreated subject (e.g., a subject untreated with an lαlp (e.g., lαl, Pαl, a heavy chain (e.g., H1, H2, H3, H4, and/or H5), a light chain (e.g., bikunin), or a combination thereof). In other instances, the baseline level of a pro-inflammatory mediator, the baseline level of VACM-1, the baseline level of ICAM-1, the baseline sedimentation rate, and/or the baseline level of an lαlp-related biomarker may be obtained from a subject having a disease or condition described herein prior to treatment (e.g., treatment with an lαlp (e.g., lαl, Pαl, a heavy chain (e.g., H1, H2, H3, H4, and/or H5), a light chain (e.g., bikunin), or a combination thereof)), e.g., for comparison to a sample from the subject after treatment. For example, lαl and/or Pαl complexes and/or other lαlp-related biomarkers can be detected and/or measured by a variety of detection methods including, for example, gas phase ion spectrometry methods, optical methods, electrochemical methods, atomic force microscopy, radio frequency methods, surface plasmon resonance, ellipsometry, and immunological methods.

### Formulations

The invention features methods for treating or reducing the likelihood of developing a disease or condition that involve administration of lαlps (e.g., lαl, Pαl, a heavy chain (e.g., H1, H2, H3, H4, and/or H5), a light chain (e.g., bikunin), or a combination thereof), a composition (e.g., a foodstuff) that includes lαlps (e.g., lαl, Pαl, a heavy chain (e.g., H1, H2, H3, H4, and/or H5), a light chain (e.g., bikunin), or a combination thereof) and a pharmaceutically acceptable excipient, carrier, or diluent, or such compositions combined with a secondary treatment, as is described herein. The compositions (e.g., a foodstuff) can be formulated for oral consumption as a solid or a liquid. The foodstuff may be prepared in the form of a food or beverage.

Liquid forms of the compositions can include suitably flavored syrups, aqueous or oil suspensions, and flavored emulsions with edible oils such as cottonseed oil, sesame oil, coconut oil, or peanut oil, as well as elixirs and similar pharmaceutical vehicles.

The foodstuff can be sterilized by conventional sterilization techniques or may be sterile filtered. Aqueous solutions can be packaged for use as is, or lyophilized. The lyophilized preparation may be combined with an aqueous or solid food prior to administration.

Pharmaceutically acceptable excipient, carriers, and diluents that may be used to prepare a foodstuff of the invention may include, but are not limited to, ion exchangers, alumina, aluminum stearate, lecithin, self-emulsifying drug delivery systems (SEDDS) such as da-tocopherol polyethylene glycol 1000 succinate, surfactants used in pharmaceutical dosage forms such as TWEEN® surfactants or other similar polymeric delivery matrices, serum proteins such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Other delivery systems can include time-release, delayed release, or sustained release formulations incorporated into a foodstuff of the invention. Such systems can avoid repeated administrations of compositions of the invention, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates, polycaprolactones, polyesteramides, polyorthoesters, polyhydroxybutyric acids, such as poly-D-(−)-3-hydroxybutyric acid (European Patent No. 133, 988), copolymers of L-glutamic acid and gamma-ethyl-L-glutamate (Sidman, K. R. et al., Biopolymers 22: 547-556), poly (2-hydroxyethyl methacrylate) or ethylene vinyl acetate (Langer, R. et al., J. Biomed. Mater. Res. 15:267-277; Langer, R. Chem. Tech. 12:98-105), and polyanhydrides.

Other examples of sustained-release formulations of a foodstuff on the invention include semi-permeable polymer matrices in the form of shaped articles, e.g., films (see, e.g., International Patent Application Publication No. WO2010002418, which is incorporated herein by reference in its entirety), or microcapsules. Delivery systems also include non-polymer systems that are: lipids including sterols such as cholesterol, cholesterol esters and fatty acids or neutral fats such as mono- di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fused implants; and the like. Specific examples include, but are not limited to: (a) erosional systems in which the agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

The proportion or concentration of an lαlp (e.g., lαl, Pαl, a heavy chain (e.g., H1, H2, H3, H4, and/or H5), a light chain (e.g., bikunin), or a combination thereof) in the composition can vary depending upon a number of factors including dosage, chemical characteristics (e.g., hydrophobicity), and the form of the composition (e.g., solid or liquid). The lαlp (e.g., lαl, Pαl, a heavy chain (e.g., H1, H2, H3, H4, and/or H5), a light chain (e.g., bikunin), or a combination thereof) may be present in the composition in a physiological proportion. Physiological proportions may be, for example, the proportions found in a person or animal that is healthy and/or the ratio of lαl and Pαl that appears naturally in human plasma. Physiological proportions are typically from between about 60% to about 80% lαl and between about 20% to about 40% Pal. However, it is to be understood that physiological proportions may vary from these ranges, for example, due to normal variation in genetic makeup of subjects.

An lαlp (e.g., lαl, Pαl, a heavy chain (e.g., H1, H2, H3, H4, and/or H5), a light chain (e.g., bikunin), or a combination thereof) or compositions thereof can have a half-life of, for example, greater than about 1, 1.5, 2, 2.5, 3, 3.5, 4, 4.5, 5, 7.5, or 10 hours. An lαlp (e.g., lαl, Pαl, a heavy chain (e.g., H1, H2, H3, H4, and/or H5), a light chain (e.g., bikunin), or a combination thereof) or compositions thereof can have a half-life of greater than about 5 hours or, preferably, greater than about 10 hours. Longer half-lives are preferred, for example, because fewer doses are required to be administered to a subject over time.

The pH of the compositions typically will be between about 3 and about 11, for example, between about 5 and 9, between about 6 and 7, or between about 7 and 8. The use of certain of the foregoing excipients, carriers, or stabilizers may result in the formation of pharmaceutical salts.

Therapeutic Methods for Treating Necrotizing Enterocolitis with an lαlp

Necrotizing enterocolitis (NEC) is an acquired inflammatory disease of the gastrointestinal tract (GIT) the predominantly effects premature infants and newborns, in which portions of the intestines undergo necrosis (i.e., tissue death). Diagnosis, such as by a medical professional, can be confirmed by methods known in the art, including radiography. Additionally, NEC can be classified depending on its severity according to the modified Bell's Classification. The initial signs of NEC include, e.g., feeding intolerance, increased gastric residuals, abdominal distension (i.e., bloating), vomiting bile, diarrhea, lethargy, shock, and bloody stools. Symptoms may progress rapidly to respiratory issues (e.g., a low respiratory rate or apnea), a low heart rate, abdominal discoloration with intestinal perforation, peritonitis, sepsis, organ failure, systemic hypotension, and death. Mortality occurs in ˜65% of cases in which sepsis and organ failure occur (Cho et al. Expert Rev. Mol. Med. 18(e12)1-17, 2016).

Risk factors for NEC, in addition to prematurity, include prolonged rupture of the membranes with amnionitis, birth asphyxia, small-for-gestational-age infants, congenital heart disease, a preceding ischemic injury (e.g., hypoxic-ischemia), bacterial infection, enteral feedings, and the exchange transfusions. Treatment of NEC ranges, depending on its severity, from a conservative therapeutic approach (e.g., with broad-spectrum antibiotics) to surgery with resection of the affected parts of the intestine. Measures, such as breastfeeding or alternatively nutrition with pasteurized human donor milk from a milk bank, administration of probiotics, avoidance of histamine type II receptor antagonists, and restrictive antibiotic treatment have been employed for the prevention of NEC.

The pathogenesis of NEC is not yet clearly understood in the art, however, its development and progression is associated with a dysregulated immune system. In particular, NEC is associated with local (e.g., in the intestinal tissues) increases in pro-inflammatory mediators, such as Toll-like receptor 4 (TLR4), nuclear factor-KB (NF-κB), tumor necrosis factor (TNF), platelet-activating factor (PAF), interleukin (IL)-18, interferon-gamma (INF-γ), IL-6, IL-8, IL-1β, and IL-17A. Low levels of counter-regulatory mechanisms, such as IL-1 receptor antagonist (IL-1Ra), TLR9, PAF-acetylhydrolase, transforming growth factor beta (TGF-β)1&2, IL-10, and regulatory T cells likely facilitate a pro-inflammatory environment in the NEC-afflicted intestine (Cho et al. Expert Rev. Mol. Med. 18(e12)1-17, 2016).

lαlps (e.g., lαl, Pαl, a heavy chain (e.g., H1, H2, H3, H4, and/or H5), a light chain (e.g., bikunin), or a combination thereof) obtained from blood and/or milk or a composition containing such proteins and a pharmaceutically acceptable excipient, diluent, or carrier, can be administered to treat NEC by any suitable route, including, for example, parenterally, by inhalation spray, topically, nasally, buccally, by oral administration, inhalation, suppository, or by injection. Administration by injection includes, for example, intravenous, intraperitoneal, subcutaneous, intracutaneous, intramuscular, intraarticular, intraarterial, intrasynovial, intrasternal, intrathecal, intralesional, and intracranial injection. In particular, the invention contemplates administering lαlps (e.g., lαl, Pαl, a heavy chain (e.g., H1, H2, H3, H4, and/or H5), a light chain (e.g., bikunin), or a combination thereof), or a composition containing such proteins, as a foodstuff. In particular, an lαlp may be administered to a subject to treat or prevent NEC in the form of a foodstuff, in which lαlp is admixed with the foodstuff.

### Purity of Inter-Alpha Inhibitor Proteins and Methods of Manufacture

An lαlp (e.g., lαl, Pαl, a heavy chain (e.g., H1, H2, H3, H4, and/or H5), a light chain (e.g., bikunin), or a combination thereof) for use in the compositions of the invention can be obtained from milk, e.g., by methods described herein, and from blood, e.g., by methods described in International Publication No. WO2005046587 and in Provisional Application Nos. 62/490,003 and 62/614,333, herein incorporated in their entirety. The milk may be from a mammal, such as a human or a domesticated ungulate (e.g., a cow, goat, sheep, buffalo, camel, donkey, horse, reindeer, and/or yak). The mammal may be a transgenic animal which expresses recombinant lαlps (e.g., lαl, Pαl, a heavy chain (e.g., H1, H2, H3, H4, and/or H5), a light chain (e.g., bikunin), or a combination thereof) that are secreted into its milk. Alternatively, lαlps (e.g., lαl, Pαl, a heavy chain (e.g., H1, H2, H3, H4, and/or H5), a light chain (e.g., bikunin), or a combination thereof, such as human lαlps) may be expressed recombinantly in and obtained from a non-human mamma in plasma and blood by methods known in the art (See, e.g., U.S. Pat. No. 9,139,641, which is incorporated herein by reference in its entirety).

In part, the lαlps can be obtained at a purity of 80% to 100% (e.g., about 80%, about 85%, about 90%, about 95%, about 96%, about 97%, about 98%, about 99%, or about 100%) from a natural source (e.g., blood and milk) and used to prepare a foodstuff of the invention (see, e.g., U.S. Pat. No. 7,932,365, which is incorporated herein by reference in its entirety).

The compositions may include any suitable lαlp, for example, lαl, Pαl, a heavy chain, a light chain, or any combination thereof. For example, the composition may include lαl, Pαl, and/or bikunin. In some cases, the composition may include lαl and Pal. The heavy chain can be H1, H2, H3, H4, or H5. The light chain can be bikunin. The lαlps can be human lαlps.

### Combination Therapies

The methods of the invention also include administering or co-administering a second treatment in addition to lαlps (e.g., lαl, Pαl, a heavy chain (e.g., H1, H2, H3, H4, and/or H5), a light chain (e.g., bikunin), or a combination thereof) or a composition (e.g., a foodstuff) thereof for the treatment of a disease or condition described herein. For example, the second treatment may include administering to the subject an anticancer agent, an anti-inflammatory agent, an anti-viral agent, an antibiotic agent, an antifungal agent, a bronchodilator, a vasopressor, a sedative, a complement inhibitor, an anti-coagulant, an immunomodulatory agent, or an agent that induces tissue repair, regeneration, or prevents cell death.

When the method includes administering a combination of lαlps (e.g., lαl, Pαl, a heavy chain (e.g., H1, H2, H3, H4, and/or H5), a light chain (e.g., bikunin), or a combination thereof), or a composition (e.g., a foodstuff) including lαlps and a pharmaceutically acceptable excipient, diluent, or carrier, and one or more second treatment agents, each agent is present at a dosage level of between about 1% to about 100%, and more preferably between about 5% to about 95%, of the dosage normally administered in a monotherapy regimen. The agent(s) of the second treatment may be administered separately, as part of a multiple dose regimen, from the lαlps (e.g., lαl, Pαl, a heavy chain (e.g., H1, H2, H3, H4, and/or H5), a light chain (e.g., bikunin), or a combination thereof) or the composition (e.g., a foodstuff) thereof. The lαlps and agent(s) of the second treatment can be administered simultaneously or sequentially in any order. Alternatively, the agent(s) of the second treatment may be part of a single dosage form, e.g., mixed together with the lαlps (e.g., lαl, Pαl, a heavy chain (e.g., H1, H2, H3, H4, and/or H5), a light chain (e.g., bikunin), or a combination thereof) in a single composition (e.g., a foodstuff).

Exemplary agents that can be administered in combination with lαlps (e.g., lαl, Pαl, a heavy chain (e.g., H1, H2, H3, H4, and/or H5), a light chain (e.g., bikunin), or a combination thereof) or compositions thereof are described below. These agents can be administered as a second therapy, e.g., as a separate therapy (e.g., in a separate formulation, e.g., administered to the patient prior to, subsequent to, or at substantially the same time as, a foodstuff containing an lαlp) or in combination (e.g., combined in a foodstuff containing an lαlp). Non-limiting examples of diseases and conditions suitable for treatment by a second treatment agent(s) described below or by a combination of the second treatment agent(s) and an lαlp-containing composition (e.g., a foodstuff with admixed lαlp) include lung diseases (e.g., acute respiratory distress syndrome (ARDS), pneumonia, community-acquired pneumonia (CAP), chronic obstructive pulmonary disease (COPD), acute and chronic neurological and neurodegenerative disorders (e.g., central nervous system (CNS) diseases (e.g., ischemia in the brain, hypoxic ischemic brain injury (e.g., neonatal), hypoxic ischemic encephalopathy, stroke (e.g., ischemic hemorrhagic stroke), Alzheimer's disease, Parkinson's disease, traumatic brain injury (TBI), neuropathic pain, and epilepsy)), sepsis, severe shock, septic shock, cancer (e.g., cancer metastasis), metabolic disorders (e.g., diabetes type I/II, cachexia), heart disease (e.g., myocardial infarction, and congestive heart failure), ischemia (e.g., ischemia/reperfusion injury), kidney disease (e.g., acute kidney injury and polycystic kidney disease, dialysis), trauma/major injury with blood loss (e.g., wound healing); tissue damage (e.g., tissue repair following tissue or organ transplantation or surgery, repair of tissue or organ damage resulting from inflammation, disease, or injury, tissue repair to reduce internal scarring, lung tissue repair (e.g., tissue repair in a subject having lung tissue damage caused by asthma, chronic obstructive pulmonary disease (COPD), bronchitis, cystic fibrosis, pneumonia, emphysema, ARDS, pneumoconiosis, lung cancer, interstitial lung disease, pulmonary fibrosis, or sarcoidosis), brain tissue repair (e.g., tissue repair in a subject having brain tissue damage caused by ischemia, hypoxia, epilepsy, TBI, hypoxic ischemic encephalopathy, or stroke), gastrointestinal tissue repair (e.g., tissue repair in a subject having gastrointestinal tissue damage caused by an autoimmune or inflammatory disease or condition (e.g., an inflammatory bowel disease, such as Crohn's disease or ulcerative colitis) or intestinal ischemia), vascular tissue repair (e.g., tissue repair in a subject having vascular tissue damage caused by inflammation or injury), muscle tissue repair, hepatic tissue repair, or cardiac tissue repair); for infection (e.g., bacterial, viral (e.g., by H1 N1, H5N1 (Avian Flu), Dengue, Zika, and others viral infections), parasitic, or fungal infections, including treatments for biodefense (e.g., against anthrax and other bioterror/emerging pathogens)); liver disease (e.g., chronic liver injury, fatty liver disease (Nonalcoholic steatohepatitis (NASH)), acute inflammatory disease (e.g., inflammatory bowel diseases, e.g., Crohn's disease), necrotizing enterocolitis (NEC), acute pancreatitis preeclampsia, preterm labor, organ transplantation and organ failure, surgery (e.g., pre- and post-surgery), autoimmune diseases (e.g., rheumatoid arthritis (RA), multiple sclerosis (MS), systemic lupus erythematosus, alopecia areata, autoimmune hemolytic anemia, autoimmune hepatitis, dermatomyositis, diabetes (type 1), juvenile idiopathic arthritis, glomerulonephritis, Graves' disease, Guillain-Barré syndrome, idiopathic thrombocytopenic purpura, myasthenia gravis, myocarditis, pemphigus/pemphigoid, pernicious anemia, polyarteritis nodosa, polymyositis, primary biliary cirrhosis, psoriasis, scleroderma/systemic sclerosis, Sjogren's syndrome, thyroiditis, uveitis, vitiligo, and granulomatosis with polyangiitis (GPA/Wegener's)), rhinitis, exposure to a toxin (e.g., anthrax related toxins (e.g., exotoxins, lethal toxin (LT), and edema toxin (ET)), meningitis, primary immunodeficiency syndrome, and acquired immunodeficiency syndrome (AIDS).

Anti-Cancer Agents

The second treatment may include an anti-cancer agent that is used to treat or reduce the symptoms of cancer. Non-limiting examples of anti-cancer agents include, cytotoxic agents, chemotherapeutic agents, growth inhibitory agents, agents used in radiation therapy, anti-angiogenesis agents, apoptotic agents, anti-tubulin agents, immunotherapeutic agents (e.g., checkpoint inhibitors, e.g., PD-1 targeting antibodies such as Nivolumab and Pembrolizumab, antibodies targeting TIM-3, LAG-3, 2B4, CD160, A2aR, BTLA, CGEN-15049, KIR, OX40, GITR, or 4-1 BB, CTLA-4 targeting antibodies such as Ipilimumab, antibodies targeting VISTA, antibodies targeting PD-L2, Gr1, or Ly6G, Rituximab, antibodies targeting PD-L1, and antibodies targeting B7-H3, B7-H4, Gal-9, or MUC1; or anti-cancer antibodies, such as Daclizumab, Basiliximab, Palivizumab, Infliximab, Trastuzumab, Gemtuzumab ozogamicin, Alemtuzumab, Ibritumomab tiuxetan, Adalimumab, Omalizumab, Tositumomab-I-131, Efalizumab, Cetuximab, Bevacizumab, Natalizumab, Tocilizumab, Panitumumab, Ranibizumab, Eculizumab, Certolizumab pegol, Golimumab, Canakinumab, Ustekinumab, Ofatumumab, Denosumab, Motavizumab, Raxibacumab, Belimumab, Brentuximab Vedotin, Pertuzumab, Ado-trastuzumab emtansine, and Obinutuzumab), and other agents known in the art to treat cancer.

Anti-Inflammatory Agents

The second treatment may include an anti-inflammatory agent that is used to treat or reduce inflammation caused by or associated with one or more of the above described diseases or conditions. Non-limiting examples of anti-inflammatory agents include corticosteroids, statins, steroids, nonsteroidal anti-inflammatory drugs, glucocorticoids, and others known the art.

Antiviral Agents

The second treatment may include an antiviral agent that is used to treat a viral infection, such as those described above and including a viral infection caused by or associated with one or more of the diseases or conditions described above. Non-limiting examples of antiviral agents include neuraminidase inhibitors (e.g., zanamivir and oseltamivir), Matrix-2 (M2) protein inhibitors (e.g., adamantine and rimantadine), permivir, ribavirin, acyclovir, ganciclovir, foscarnet, cidofovir, and others known in the art.

Antibiotic Agents

The second treatment may include an antibiotic agent that is used to treat a bacterial infection, such as those described above and including a bacterial infection caused by or associated with one or more of the diseases or conditions described above. Non-limiting examples of antibiotic agents include amoxicillin, penicillin, doxycycline, clarithromycin, benzylpenicillin, azithromycin, daptomycin, linezolid, levofloxacin, moxifloxacin, gatifloxcin, gentamicin, macrolides, cephalosporins, azithromycin, ciprofloxacin, cefuroxime, amoxillin-potassium clavulanate, erythromycin, sulfamethoxazole-trimethoprim, doxycycline monohydrate, cefepime, ampicillin, cefpodoxime, ceftriaxone, cefazolin, erythromycin ethylsuccinate, meropenem, piperacillin-tazobactam, amikacin, erythromycin stearate, cefepime in dextrose, doxycycline hyclate, ampicillin-sulbactam, ceftazidime, gemifloxacin, gentamicin sulfate, erythromycin lactobionate, imipenem-cilastatin, cefoxitin, cefditoren pivoxil, ertapenem, doxycycline-benzoyl peroxide, ampicillin-sulbactam, meropenem, cefuroxime, cefotetan, piperacillin-tazobactam, broad-spectrum fluoroquinolones (which may be used, for example, to treat pneumonia caused by atypical pathogens such as Mycoplasma pneumoniae or Chlamydophila pneumoniae), and others known in the art.

Antifungal Agents

The second treatment may include an antifungal agent that is used to treat a fungal infection, e.g., a fungal infection caused by or associated with one or more of the diseases or conditions described above. Non-limiting examples of antifungal agents include amphotericin, caspofungin, voriconazole, itraconazole, posaconazole, fluconazole, flucytosine, and others known in the art.

Antiparasitic Agents

The second treatment may include an antiparasitic agent that is used to treat a parasitic infection (e.g., a parasitic protozoan infection), such as, a parasitic infection caused by or associated with one or more of the diseases or conditions described above. Non-limiting examples of antiparasitic agents include nitazoxanide, melarsoprol, eflornithine, metronidazole, tinidazole, miltefosine, mebendazole, pyrantel pamoate, thiabendazole, diethylcarbamazine, ivermectin, albendazole, praziquantel, rifampin, and others known in the art.

Bronchodilators

The second treatment may include a bronchodilator that is used to relax the bronchial muscles allowing airways to be larger and air to pass through the lungs. A bronchodilator can be administered in combination with an lαlp to treat a respiratory disease or condition, such as one or more of the respiratory conditions or diseases described herein or caused by or associated with one or more of the diseases or conditions described herein. Non-limiting examples of bronchodilators include beta 2 agonists, xanthines, ipratropium, oxitropium, muscarinic receptor antagonists, ipratropium, oxitropium, theophylline, theobromine, caffeine, salbutamol, isoproterenol, albuterol, levalburerol, pirbuterol, metaproterenol, terbutaline, salmeterol, formoterol, and others known in the art.

Vasopressors

The second treatment may include a vasopressor that causes vasoconstriction and/or an increase in blood pressure. A vasopressor can be administered in combination with an lαlp to treat a blood pressure disease or condition, such as one or more of the blood pressure conditions or diseases described herein or caused by or associated with one or more of the diseases or conditions described herein. Non-limiting examples of vasopressors include epinephrine, isoproterenol, phenylephrine, norepinephrine, dobutamine, ephedrine, droxidopa, and others known in the art.

Sedatives

The second treatment may include a sedative. A sedative can be administered in combination with an lαlp to treat, e.g., anxiety, such as anxiety related to painful or anxiety-provoking medical procedures, associated with a disease or condition described herein. Sedatives may also be administered to improve tractability and compliance of, e.g., children. Non-limiting examples of sedatives include propofol, diprivan, morphine, fentanyl, midazolam, lorazepam, precede, infumorph, dexmedetomidine, alfentanil, and others known in the art.

Complement Inhibitors

The second treatment may include an inhibitor of complement activation. A complement inhibitor can be administered in combination with an lαlp to treat an inflammatory and/or degenerative disease or condition, such as one or more of the inflammatory and/or degenerative diseases or conditions described herein or caused by or associated with one or more of the diseases or conditions described herein. The composition may inhibit activation of one or more complement components such as C1, C2, C3 (e.g., C3a and C3b), C4 (e.g., C4b), C5 (e.g., C5a and C5b), C6, C7, C8, C9, membrane attack complex, Factor B, Factor D, MASP-1, and MASP-2, or fragments thereof. The complement inhibitors may include protease inhibitors such as C1-INH and Rhucin/rhC11 NH, soluble complement regulators such as sCR1/TP10, CAB-2/MLN-2222, therapeutic antibodies such as eculizumab/SOLIRIS®, Pexelizumna, ofatumumab, complement component inhibitors such as compstatin, receptor antagonists such as PMX-53 and rhMBL.

Anti-Coagulants

The second treatment may include an anti-coagulant that works to prevent blood coagulation (i.e., clotting). An anti-coagulant can be administered in combination with an lαlp to treat a heart or circulatory system disease or condition, such as one or more of the heart or circulatory system diseases or conditions described herein or caused by or associated with one or more of the diseases or conditions described herein. Non-limiting examples of anti-coagulants include coumarins (i.e., vitamin K antagonists, e.g., warfarin (COUMADIN®)), heparins, thrombin inhibitors, anti-thrombin III, inhibitors of factor IIa (DABIGATRAN®), inhibitors of factor Xa (RIVAROXABAN®, APIXABAN®, and EDOXABAN®), activated Protein C, and protease inhibitors, such as furin inhibitors, and others known in the art.

Immunomodulatory Agents

The second treatment may include immunomodulatory agents (IMiDs) that work, e.g., by stimulating natural killer cells and activating T cells. An IMiD can be administered in combination with an lαlp to treat a cancer or an autoimmune disease or condition, such as one or more of the cancers or autoimmune diseases or conditions described herein or caused by or associated with one or more of the diseases or conditions described herein. Non-limiting examples of immunomodulators include interleukins (e.g., IL-2, IL-7, IL-12), cytokines (e.g., interferons, G-CSF, and Imiquimod), chemokines (e.g., CCL3, CCL26, CXCL7), IMiDs (e.g., thalidomide (THALOMID®), lenalidomide (REVLIMID®), bortezomib (VELCADE®), and pomalidomide (POMALYST®)), cytosine phosphate-guanosine, oligodeoxynucleotides, glucans, and others known in the art.

Agents that Induce Tissue Repair

The second treatment may include an agent that induces tissue repair, regeneration, or prevents cell death. An agent that can induce tissue repair can be administered in combination with an lαlp to treat tissue damage, such as tissue damage associated with or caused by one or more of the diseases or conditions described herein. Non-limiting examples of such agents include stem cells, collagens, fibronectins, laminins, integrins, angiogenic factors, anti-inflammatory factors, glycosaminoglycans, vitrogen, antibodies and fragments thereof, functional equivalents of these agents, and combinations thereof.

Anticholinergics

The second treatment may include an anticholinergic (e.g., antimuscarinic agents, ganglionic blockers, and neuromuscular blockers) that work, e.g., by blocking the binding of the neurotransmitter acetylcholine to its receptor within the tissues (e.g., nerve cells) of the central and peripheral nervous system, thereby inhibiting parasynthetic nerve impulses that are responsible for the involuntary movement (e.g., contraction) of smooth muscles, e.g., muscle in the gastrointestinal (GI) tract, urinary tract, lungs, and other body parts. An anticholinergic can be administered in combination with an lαlp to treat a gastrointestinal or respiratory disease or condition, such as one or more of the gastrointestinal or respiratory diseases or conditions described herein or caused by or associated with one or more of the diseases or conditions described herein.

Antidiarrheals

The second treatment may include an antidiarrheal that provides symptomatic relief for diarrhea and the symptoms thereof. An antidiarrheal may include electrolyte solutions used to replace lost fluids and salts; bulking agents like methylcellulose, guar gum, or a plant fiber (e.g., bran, sterculia, isabgol, and others); absorbents that absorb toxic substances that cause infective diarrhea (e.g., methylcellulose); anti-inflammatory compounds such as bismuth subsalicylate; anticholinergics that reduce intestinal movement, diarrhea, and accompanying cramping; and opioids (e.g., morphine, codeine, and loperamide). An antidiarrheal can be administered in combination with an lαlp to treat a gastrointestinal disease or condition, such as one or more of the gastrointestinal diseases or conditions described herein or caused by or associated with one or more of the diseases or conditions described herein.

Antidepressants

The second treatment may include an antidepressant (e.g., selective serotonin reuptake inhibitors (SSRIs), serotonin-norepinephrine reuptake inhibitors (SNRIs), tricyclic antidepressants (TCAs), monoamine oxidase inhibitors (MAOIs), reversible monoamine oxidase A inhibitors (rMAO-A inhibitors), tetracyclic antidepressants (TeCAs), and noradrenergic and specific serotonergic antidepressant (NaSSAs)). An antidepressant can be administered in combination with an lαlp to treat pain caused by or associated with one or more of the diseases or conditions described herein. Non-limiting examples of TCAs include amitriptyline, butriptyline, clomipramine, desipramine, dosulepin, doxepin, imipramine, iprindole, lofepramine, nortriptyline, protriptyline, and trimipramine.

Prokinetic Agents

The second treatment may include a prokinetic agent (e.g., a gastroprokinetic agent) that enhances gastrointestinal (GI) motility (e.g., movement of food through the digestive system and out of the body) by increasing the frequency of muscle contractions in the small intestines and/or by strengthening the muscle contractions of the small intestine (e.g., without disrupting the normal rhythm of contraction). A prokinetic agent can be administered in combination with an lαlp to treat a gastrointestinal disease or condition, such as one or more of the gastrointestinal diseases or conditions described herein or caused by or associated with one or more of the diseases or conditions described herein. Non-limiting examples of prokinetic agents include benzamide, cisapride, domperidone, erythromycin, itopride, mosapride, metoclopramide, prucalopride, renzapride, tegaserod, mitemcinal, levosulpiride, and cinitapride.

Laxatives

The second treatment may include a laxative (e.g., a bulk-forming laxative, an emollient agent, lubricant agents, hyperosmotic agents, saline laxative agents, stimulant agents, oil-based agents, serotonin agonists, and chloride channel activators) that loosen stools, increase bowel movements, and reduce constipation. A laxative can be administered in combination with an lαlp to treat a gastrointestinal disease or condition, such as one or more of the gastrointestinal diseases or conditions described herein or caused by or associated with one or more of the diseases or conditions described herein. Non-limiting examples of bulk-forming laxative include psyllium (METAMUCIL®), polycarbophil (FIBERCON®), and methylcellulose (CITRUCEL®).

Neurotransmitters

The second treatment may include a neurotransmitter, such as serotonin, glutamate, GABA, acetylcholine, dopamine, norepinephrine, epinephrine, histamine, and others. Additionally, drugs that alter neurotransmitter activity (e.g., drugs that increase and/or decrease the rate of synthesis of neurotransmitters, such as precursors), drugs that alter neurotransmitter storage in synaptic vesicles, drugs that alter neurotransmitter binding to target receptors (e.g., receptor agonists and/or antagonists), drugs that interfere with neurotransmitter deactivation, and drugs that block neuronal activity are also contemplated as useful secondary treatments for use in the methods of the invention, e.g., a neurotransmitter can be administered in combination with an lαlp to treat neuropathic pain caused by or associated with one or more of the diseases or conditions described herein.

Antispasmodics The second treatment may include an anti-spasmodic that suppresses muscles spasms (e.g., contractions) of e.g., the stomach, intestines, urinary tract, and bladder. Non-limiting examples of antispasmodics include receptor antagonists, chloride channel activators, and guanylate cyclase C (GC-C) agonists, and pain relievers. An antispasmodic can be administered in combination with an lαlp to treat a gastrointestinal disease or condition, such as one or more of the gastrointestinal diseases or conditions described herein or caused by or associated with one or more of the diseases or conditions described herein.

Pain Relievers

The second treatment may include an analgesic or pain reliever, such as over-the-counter (OTC) or a prescribed pain reliever. A pain reliever can be administered in combination with an lαlp to treat pain caused by or associated with one or more of the diseases or conditions described herein. Non-limiting examples of pain relievers include acetaminophen (e.g., TYLENOL®) and nonsteroidal anti-inflammatory drugs (NSAIDS) (e.g., aspirin, naproxen (ALEVE®), ibuprofen (e.g., ADVIL®, MOTRIN®), and others), COX-2 inhibitors (e.g., rofecoxib, celecoxib, and etoricoxib), opioids (e.g., morphine, codeine, oxycodone, hydrocodone, dihydromorphine, and pethidine), psychotropic agents (e.g., ketamine, clonidine, α2-adrenoreceptor agonists, mexiletine, and other local anesthetic analogues), and medical cannabis, and others.

Methods of Purification

The invention features methods of purifying an lαlp (e.g., lαl, Pαl, a heavy chain (e.g., H1, H2, H3, H4, and/or H5), a light chain (e.g., bikunin), or a combination thereof) from milk by separating a fraction of milk comprising the lαlp, and purifying the lαlp from the fraction of milk, wherein the lαlp has with a purity ranging from about 85 to about 100% (e.g., 85, 86, 87, 88, 89, 90, 91, 92, 93, 93, 94, 95, 96, 97, 98, or 100% pure). A fraction of milk can be prepared, e.g., by a decanting step, a clarification step, a chromatography step, a centrifugation and/or sedimentation step, a freezing step, a drying, an evaporation step, an extraction step, a filtration step, a precipitation step, or by other methods known in the art. The method includes separating lαlps from milk proteins to produce a purified lαlp preparation. For example, lαlps (e.g., lαl, Pαl, a heavy chain (e.g., H1, H2, H3, H4, and/or H5), a light chain (e.g., bikunin), or a combination thereof) may be separated from milk proteins present in the milk fraction using one or more chromatography steps. The chromatography steps may include anion-exchange chromatography, affinity chromatography, and/or size-exclusion chromatography. Additional purification steps used to obtain a purified lαlp preparation may include solid phase extraction, and/or precipitation (e.g., a methanol-chloroform precipitation). The precipitation step can include exposure to low pH (e.g., a pH between about 4.2 and about 5.2, such as a pH of 4.2, 4.3, 4.4, 4.5, 4.6, 4.7, 4.8, 4.9, 5.0, 5.1, or 5.2) to remove casein from the lαlp preparation. The purification method may also include the step of exposing the lαlp preparation to a pH of about 5.5 or lower (e.g., a pH of about 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.1, 3.0, 2.9, 2.8, 2.7, 2.6, 2.5. 2.4, 2.3, 2.2, 2.1, or about 2.0), such as washing a chromatography support to which lαlp is bound with a wash buffer having a pH of 5.5 or lower.

A method for purifying an lαlp from milk (e.g., human milk or milk from a domesticated ungulate) may include a centrifugation step, after which the fat layer may be removed and the supernatant collected. The supernatant may then be filtered, and optionally diluted, prior to application to a support for chromatographic separation. After the supernatant is applied to the support, the support may be washed using one or more wash buffers (e.g., a salt-containing buffer and, optionally, a low pH buffer, such as a buffer having a pH of about 5.5 or lower (e.g., a pH of about 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.1, 3.0, 2.9, 2.8, 2.7, 2.6, 2.5. 2.4, 2.3, 2.2, 2.1, or about 2.0)). lαlp can then be eluted from the column using an elution buffer. The pH of the milk or supernatant may be adjusted prior to the centrifugation and/or chromatography steps. The milk may be obtained from any mammal, such as a human or domesticated ungulate (e.g., a cow, goat, sheep, buffalo, camel, donkey, horse, reindeer, and yak). The lαlp may be a human lαlp expressed recombinantly in the mammal (e.g., the mammal may be a transgenic mammal engineered to express human lαlp).

The method may also include the further steps of evaluating and/or determining the biochemical and/or biophysical properties of the purified lαlp (e.g., lαl, Pαl, a heavy chain (e.g., H1, H2, H3, H4, and/or H5), a light chain (e.g., bikunin), or a combination thereof) by any appropriate method known in the art.

The purified lαlp has an apparent molecular weight of between about 60 kDa to about 280 kDa, which can be determined by any appropriate method known in the art, e.g., by sodium dodecyl sulfate polyacrylamide gel electrophoresis.

Additionally, the purified lαlps exhibit a half-life of one hour or greater (e.g., 1, 2, 3, 4, 5, 6, 7 8, 9, 10, 11, 12, 13, 14, 15, 16, 17, 18, 19, 20 hours).

The lαlps should also have a biological activity, such as an activity selected from the group consisting of a cytokine inhibitor activity, increase of cytokine activity, chemokine inhibitor activity, protease inhibitor activity (e.g., serine protease inhibitor activity), chondroitin sulfate binding, glycosaminoglygan binding activity, hyaluronic acid binding activity, complement binding activity, histone binding activity, Arg-Gly-Asp (RGD) domain binding activity, coagulation factor binding activity, cellular repair activity, and extracellular matrix protein binding activity. The lαlps also exhibit a high trypsin inhibitory specific activity, e.g., between about 1000 IU/mg to about 2000 IU/mg (e.g., 1000, 1100, 1200, 1300, 1400, 1500, 1600, 1700, 1800, 1900, or 2000 IU/mg).

An lαlp (e.g., a human lαlp) may also be obtained and purified from a mammal (e.g., a domesticated ungulate (e.g., a cow, goat, sheep, buffalo, camel, donkey, horse, reindeer, and yak)) that has been genetically modified to express the lαlp according to the methods described above. The mammal may contain milk-producing cells transfected with a transgene that includes a nucleic acid sequence encoding an lαlp, a milk-specific promoter, said promoter being operably linked to the nucleic acid sequence encoding the lαlp, and a leader sequence encoding a protein secretory signal that enables secretion of the lαlp by the milk-producing cell.

### Kits

The invention also features kits containing a foodstuff (e.g., a liquid or a solid foodstuff) including an lαlp and/or a composition of lαlp suitable for admixing with a foodstuff. For example, the kit may include a foodstuff (e.g., a liquid or solid foodstuff) and a separate container of an lαlp (e.g., a composition of an lαlp suitable for oral consumption). The kit may also include a separate container of a pharmaceutically acceptable excipient, carrier, and/or diluent that may be used in preparing a foodstuff of the invention (e.g., a liquid). For example, the kit may contain a beverage in a container with a lyophilized or powder form of lαlp that can be added to the beverage prior to consumption.

The kit may also include a recipe for making a suitable foodstuff (e.g., a liquid or solid foodstuff), and instructions for admixing an lαlp (e.g., a composition of an lαlp suitable for oral consumption) with a foodstuff, and, optionally, instructions for therapeutic use.

A kit of the invention may also include an additional therapeutic agent, e.g., an anticancer agent, an anti-inflammatory agent, an anti-viral agent, an antibiotic agent, an antifungal agent, a bronchodilator, a vasopressor, a sedative, a complement inhibitor, an anti-coagulant, and an immunomodulatory agent. The additional therapeutic agent may be provided in a separate container, as a separate formulation, or admixed with the foodstuff containing an lαlp.

Assays

Therapeutic efficacy of a foodstuff of the invention may be monitored, e.g., by methods known in the art, to determine pre- and post-treatment levels of lαlps (e.g., lαl, Pαl, a heavy chain (e.g., H1, H2, H3, H4, and/or H5), a light chain (e.g., bikunin), or a combination thereof), pro-inflammatory mediators, VCAM-1, ICAM-1, and lαlp-related biomarkers (e.g., histone, extracellular histone, histone/Pal complexes, histone/Ial complexes, histone lαl/Pαl complexes, TNF-α, IL-6, IL-10, IL-1, IL-1ra, IL1B, IL-8, MCP-1, MIP-2, C-reactive protein (CRP), procalcitonin (PCT), cytokine-induced neutrophil chemoattractant/KC, UTI, complement components C1, C2, C3, C4, C5, C6, C7, C8, C9, membrane attack complex, Factor B, Factor D, MASP-1, and MASP-2, or fragments thereof) associated with disease progression and/or resolution. Therapeutic efficacy of a foodstuff of the invention may also be monitored using assays that evaluate the severity of inflammation, such as measurements of sedimentation rate (erythrocyte sedimentation rate). The level of lαlps, pro-inflammatory mediators, VCAM-1, ICAM-1, and lαlp-related biomarkers can be detected and/or measured e.g., by gas phase ion spectrometry methods, optical methods, electrochemical methods, atomic force microscopy, radio frequency methods, surface plasmon resonance, ellipsometry, and immunological methods. Sedimentation rate can be measured using standard clinical tests (e.g., blood tests).

An immunoassay can be used to detect and analyze lαlps (e.g., lαl, Pαl, a heavy chain (e.g., H1, H2, H3, H4, and/or H5), a light chain (e.g., bikunin), or a combination thereof) and/or other biomarker protein levels in a sample. The immunoassay can include: (a) providing an antibody that specifically binds to lαl and/or Pαl; (b) contacting a sample with the antibody; and (c) detecting the presence of a complex of the antibody bound to the proteins in the sample. Suitable antibodies for use in an immunoassay to detect lαlps include, MAb 69.31, MAb 69.26, anti-lαlp polyclonal antibody, and anti-bikunin monoclonal or polyclonal antibody.

The following examples are intended to illustrate, rather than limit, the invention.

### EXAMPLES

### Example 1

### Administration of a Foodstuff Comprising an lαlp to a Human Subject Having Necrotizing Enterocolitis

A foodstuff containing an lαlp can be administered to a human subject, such as a premature infant, having or being at risk of having necrotizing enterocolitis.

For example, a premature infant identified as having or being at risk of having necrotizing enterocolitis may be administered PEDIALYTE® or cow's milk containing a therapeutically effective dose of an lαlp (e.g., a human lαlp, such as lαl, Pαl, and/or bikunin), or a combination thereof), such as between about 10 mg/kg to about 30 mg/kg. The infant can be administered one or more doses of the foodstuff until symptoms improve.

The infant can be monitored for presentation or resolution of symptoms. If necessary, additional doses of the foodstuff can be administered. As the condition of the infant improves, the frequency of the therapy may decrease to one or more times every day.

### Example 2

### Administration of a Foodstuff Comprising an lαlp to a Human Subject Having Crohn's Disease

A foodstuff containing an lαlp can be administered to a human patient diagnosed with and receiving treatment for an inflammatory bowel disease, e.g., Crohn's disease. For example, the patient may already be receiving treatment, e.g., with anti-inflammatory drugs (e.g., oral 5-aminosalicylates and/or corticosteroids), immunomodulatory drugs (e.g., azathioprine, methotrexate, infliximab, adalimumab, certolizumab, and/or natalizumab) and/or antibiotics (e.g., metronidazole and/or ciprofloxacin). The patient can be administered an electrolyte solution containing a therapeutically effective dose of an lαlp (e.g., lαl, Pαl, and/or bikunin), or a combination thereof), such as between about 10 mg/kg to about 30 mg/kg. The patient can be administered one or more doses of the electrolyte solution until symptoms improve.

The patient can be monitored for presentation or resolution of symptoms. If necessary, additional doses of the electrolyte solution can be administered. As the condition of the patient improves, the frequency of the therapy may decrease to one or more times every day, every other day, every week, or as needed (e.g., prior to consuming a regular meal).

### Example 3

### Administration of a Foodstuff Comprising an lαlp to a Human Subject Having an Inflammatory Disease

A foodstuff containing an lαlp can be administered to a human patient for inflammation, e.g., due to a wound, after it was determined that the patient had low levels of an lαlp in their blood.

For example, the patient may be administered a protein bar containing a therapeutically effective dose of an lαlp (e.g., lαl, Pαl, and/or bikunin), or a combination thereof), such as between about 10 mg/kg to about 30 mg/kg. The patient can be administered one or more doses of the foodstuff until the level of the lαlp in their blood has sufficiently increased. The patient can be monitored for presentation or resolution of symptoms. If necessary, additional doses of the foodstuff can be administered. As the condition of the patient improves, the frequency of the therapy may decrease to one or more times every day, every other day, every week, or as needed (e.g., prior to consuming a regular meal).

### Example 4

### Administration of a Foodstuff Comprising an lαlp to a Human Subject Having a Respiratory Disease

A foodstuff containing an lαlp can be administered to a human patient diagnosed with and receiving treatment for acute respiratory distress syndrome (ARDS).

For example, the patient can have inflammation caused, e.g., by sepsis, endothelial dysfunction, fluid leakage from the capillaries, and/or impaired drainage of fluid from the lungs, and may be administered an electrolyte solution containing a therapeutically effective dose of an lαlp (e.g., lαl, Pαl, and/or bikunin), or a combination thereof), such as between about 10 mg/kg to about 30 mg/kg. The patient can be administered one or more doses of the foodstuff until symptoms improve.

The patient can be monitored for presentation or resolution of symptoms. If necessary, additional doses of the foodstuff can be administered. As the condition of the patient improves, the frequency of the therapy may decrease, e.g., to one or more times every day.

### Example 5

### Administration of a Foodstuff Comprising an lαlp to a Human Subject Having Ischemia

A foodstuff containing an lαlp can be administered to a human subject, such as an infant having ischemia, e.g., hypoxic-ischemic encephalopathy due to birth asphyxia.

For example, an infant identified as having hypoxic-ischemic encephalopathy may be administered an infant formula containing a therapeutically effective dose of an lαlp (e.g., lαl, Pαl, and/or bikunin), or a combination thereof), such as between about 10 mg/kg to about 30 mg/kg. The infant can be administered one or more doses of the foodstuff until symptoms improve.

The infant can be monitored for presentation or resolution of symptoms. If necessary, additional doses of the foodstuff can be administered. As the condition of the infant improves, the frequency of the therapy may decrease, e.g., to one or more times every day.

### Example 6

### Administration of a Foodstuff Comprising an lαlp to a Human Subject Having Sepsis

A foodstuff containing an lαlp can be administered to a human patient diagnosed with and receiving treatment for sepsis, e.g., severe sepsis causing poor organ function.

For example, the patient may already be receiving treatment, e.g., with broad-spectrum antibiotics (e.g., two or more β-lactam antibiotics) and may be administered an oral rehydration solution containing a therapeutically effective dose of an lαlp (e.g., lαl, Pαl, and/or bikunin), or a combination thereof), such as between about 10 mg/kg to about 30 mg/kg. The patient can be administered one or more doses of the foodstuff until symptoms improve.

The patient can be monitored for presentation or resolution of symptoms. If necessary, additional doses of the foodstuff can be administered. As the condition of the patient improves, the frequency of the therapy may decrease, e.g., to one or more times every day.

### Example 7

### Administration of a Foodstuff Comprising an lαlp to a Human Subject Having a Viral Infection

A foodstuff containing an lαlp can be administered to a human subject diagnosed with and receiving treatment for a viral infection e.g., an influenza virus infection, e.g., an avian flu infection, e.g., an H5N1 infection, due to exposure and/or contact with birds.

For example, the patient may already be receiving treatment, e.g., with an anti-viral (e.g., a neuraminidase inhibitor, such as oseltamivir or zanamivir) and may be administered an oral rehydration solution containing a therapeutically effective dose of an lαlp (e.g., lαl, Pαl, and/or bikunin), or a combination thereof), such as between about 10 mg/kg to about 30 mg/kg. The patient can be administered one or more doses of the foodstuff until symptoms improve.

The patient can be monitored for presentation or resolution of symptoms. If necessary, additional doses of the foodstuff can be administered. As the condition of the patient improves, the frequency of the therapy may decrease, e.g., to one or more times every day.

### Example 8

### Extraction of lαlp from Milk

Bovine or human milk was adjusted to pH 4.5 using Acetic acid and centrifugated at 7500 rpm for 15 min. Supernatant was collected after the fat layer was removed. The pH of the milk supernatant was then adjusted to pH 6.8 by adding 3 M Tris buffer. Following filtration, the milk supernatant was diluted 1:3 with buffer containing 20 mM Tris, pH 7.5 and 150 mM NaCl for further chromatographic separation. The diluted milk was then applied to a Tosoh GigaCap Q-650 column. Following collection of the unbound fraction (flow-through), the column was initially washed with buffer containing salt (20 mM Tris, pH 7.5+300 mM NaCl) and subsequently washed with low pH buffer (50 mM Acetic acid, pH 4.5). The wash fractions were collected and the column was then eluted using elution buffer containing 20 mM Tris, pH 7.5 and 750 mM NaCl.

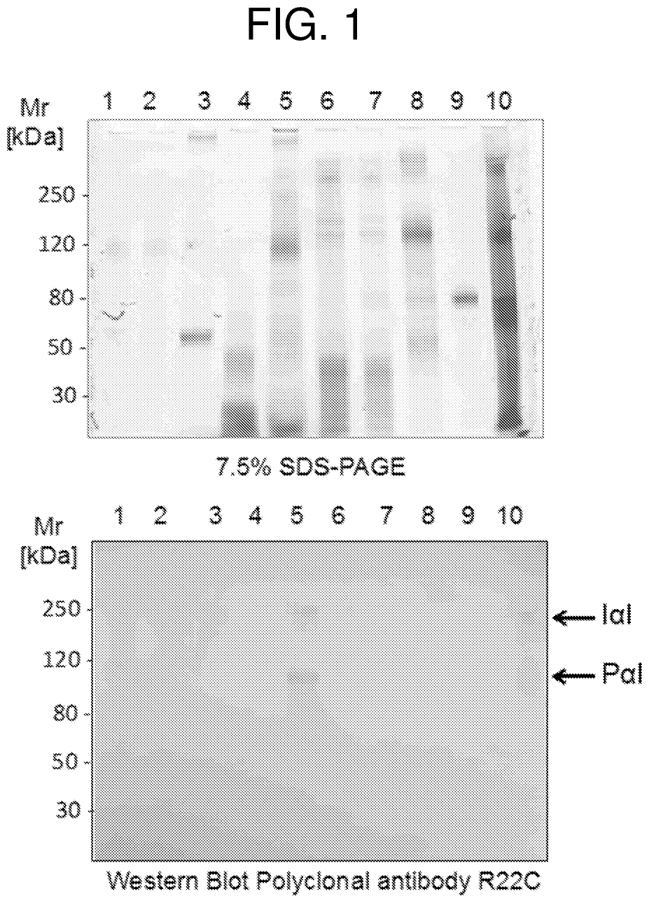

The aliquots of the unbound, wash fractions, and eluted fraction were separated on 7.5% SDS-PAGE gels (BioRad TGX gels) and subsequently transferred onto nitrocellulose membrane for western blot analysis. Following blocking with 5% non-fat dried milk, the nitrocellulose membrane was incubated with biotinylated-rabbit polyclonal antibody against rat IAIP (R22C). This polyclonal antibody cross-reacts with human and bovine lαlp. Following several washes with PBS+0.05% Tween, the membrane was incubated with Streptavidin-HRP and the reactivity was visualized using the metal enhanced DAB substrate (Pierce). As shown in FIG. 1, bovine and human milk-derived lαlp were bound to the column and eluted from the column using 750 mM NaCl. Specific bands of 250 kDa and 125 kDa, corresponding to lαl and Pαl, respectively, of bovine and human milk derived lαlp were detected by the rabbit polyclonal antibody R22C (Lanes 5 and 10, arrows).

### Example 9

### Administration of a Foodstuff Comprising an lαlp to a Human Subject Having Lung Tissue Damage

A foodstuff containing an lαlp can be administered to a human patient with lung tissue damage, e.g., due to asthma, COPD, bronchitis, cystic fibrosis, pneumonia, emphysema, ARDS, pneumoconiosis, lung cancer, interstitial lung disease, pulmonary fibrosis, or sarcoidosis, e.g., to promote or increase lung tissue repair, after it was determined that the patient had low levels of an lαlp in their blood.

For example, the patient may be administered a protein bar containing a therapeutically effective dose of an lαlp (e.g., lαl, Pαl, and/or bikunin), or a combination thereof), such as between about 10 mg/kg to about 30 mg/kg. The patient can be administered one or more doses of the foodstuff until the level of the lαlp in their blood has sufficiently increased.

The patient can be monitored for presentation or resolution of symptoms. If necessary, additional doses of the foodstuff can be administered. As the condition of the patient improves, the frequency of the therapy may decrease to one or more times every day, every other day, every week, or as needed (e.g., prior to consuming a regular meal).

### Other Embodiments

All publications, patents, and patent applications mentioned in the above specification are hereby incorporated by reference to the same extent as if each individual publication, patent or patent application was specifically and individually indicated to be incorporated by reference in its entirety. Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A foodstuff comprising an inter-alpha inhibitor protein (lαlp), wherein the lαlp is present in the foodstuff in an amount of at least about 0.01 milligram per mg of the foodstuff.

2. The foodstuff of claim 1, wherein the lαlp is lαl, Pαl, H1, H2, H3, H4, H5, bikunin, or a combination thereof.

3. The foodstuff of claim 2, wherein the lαlp comprises lαl, Pαl, and/or bikunin.

4. The foodstuff of claim 2 or 3, wherein the lαlp comprises H1, H2, H3, H4, and/or H5.

5. The foodstuff of any one of claims 2-4, wherein the lαlp comprises bikunin.

6. The foodstuff of any one of claims 1-5, wherein the lαlp admixed with the foodstuff ranges in purity from about 85% to about 100% pure.

7. The foodstuff of any one of claims 1-6, wherein the lαlp is present in the foodstuff in an amount of about 0.1 milligram (mg) to about 10 mg per mg of the foodstuff.

8. The foodstuff of any one of claims 1-6, wherein the lαlp is present in the foodstuff in an amount of about 10 mg to about 1000 mg per liter (L) of the foodstuff.

9. The foodstuff of any one of claims 1-8, wherein the lαlp is isolated from blood or milk.

10. The foodstuff of claim 9, wherein the blood or milk is from a mammal.

11. The foodstuff of claim 10, wherein the mammal is a domesticated ungulate.

12. The foodstuff of claim 11, wherein the domesticated ungulate is selected from the group consisting of a cow, goat, sheep, buffalo, camel, donkey, horse, reindeer, and yak.

13. The foodstuff of any one of claims 10-12, wherein the lαlp is expressed recombinantly in the mammal.

14. The foodstuff of claim 13, wherein the lαlp is a human lαlp.

15. The foodstuff of any one of claims 1-14, wherein the lαlp has an apparent molecular weight of between about 60 kDa to about 280 kDa.

16. The foodstuff of claim 15, wherein the molecular weight is determined by sodium dodecyl sulfate polyacrylamide gel electrophoresis.

17. The foodstuff of any one of claims 1-16, wherein the lαlp has an in vivo half-life of greater than one hour.

18. The foodstuff of claim 17, wherein the in vivo half-life is greater than five hours.

19. The foodstuff of any one of claims 1-18, wherein the lαlp has a biological activity.

20. The foodstuff of claim 19, wherein the biological activity is selected from the group consisting of a cytokine inhibitor activity, increase of cytokine activity, chemokine inhibitor activity, protease inhibitor activity, chondroitin sulfate binding, glycosaminoglygan binding activity, hyaluronic acid binding activity, complement binding activity, histone binding activity, Arg-Gly-Asp (RGD) domain binding activity, coagulation factor binding activity, cellular repair activity, and extracellular matrix protein binding activity.

21. The foodstuff of any one of claims 1-20, wherein the lαlp has a high trypsin inhibitory specific activity.

22. The foodstuff of claim 21, wherein the trypsin inhibitory specific activity is between about 1000 IU/mg to about 2000 IU/mg.

23. The foodstuff of any one of claims 1-22, wherein the foodstuff is pasteurized at a dry heat between about 50° C. and about 120° C.

24. The foodstuff of any one of claims 1-23, wherein the foodstuff comprises at least one pharmaceutically acceptable excipient, diluent, carrier, and/or stabilizer.

25. The foodstuff of claim 24, wherein the stabilizer is selected from the group consisting of albumin, polyethylene glycol, alpha-trehalose, amino acids, salts, glycerol, omega-amino acids, sugar, and combination thereof.

26. The foodstuff of any one of claims 1-25, wherein the foodstuff is selected from the group consisting of a beverage, a milk-based product, a baked good, a fruit and/or vegetable-based product, a grain and/or cereal-based product, a non-dairy product, an infant formula, an electrolyte product, a sports drink, a protein-based product, a nutritional supplement, a food additive, a flavoring, a sweetener, a preservative, a food coloring agent, and a fiber.

27. The foodstuff of claim 26, wherein the milk-based product is selected from the group consisting of milk, cream, butter, yogurt, kefir, ice cream, gelato, sherbet, custard, pudding, nougat, cheese, a whey product, and a casein product.

28. The foodstuff of claim 26, wherein the baked good is selected from the group consisting of a biscuit, bread, brownie, cake, casserole, cookie, cracker, pastry, pie, pizza, and tart.

29. The foodstuff of claim 26, wherein the fruit and/or vegetable-based product is selected from the group consisting of an oil, jelly, jam, marmalade, preserve, butter, puree, infant food, sauce, soup, and broth.

30. The foodstuff of claim 26, wherein the non-dairy product is selected from the group consisting of a cheese substitute, non-dairy yogurt, non-dairy cream, non-dairy butter, non-dairy ice cream, non-dairy milk, tofu, soy-based product, nut-based product, coconut-based product, and gelatin.

31. The foodstuff of claim 26, wherein the cereal-based product is selected from the group consisting of bread, pasta, oatmeal, breakfast cereal, tortilla, and grits.

32. The foodstuff of claim 26, wherein the infant formula is selected from the group consisting of a protein hydrolysate formula, metabolic formula, amino acid based formula, exempt infant formula, specialized formula, follow-on formula, and a toddler formula.

33. The foodstuff of claim 26, wherein the electrolyte product is selected from the group consisting of a pre-mixed solution, a dissolvable tablet, an edible gel, a concentrated solution, and a powder.

34. The foodstuff of claim 26, wherein the electrolyte product and/or the sports drink is selected from the group consisting of an isotonic, hypertonic, and hypotonic solution.

35. The foodstuff of claim 26, wherein the nutritional supplement and/or protein-based product is selected from the group consisting of a meal replacement product, protein or nutritional shake, protein bar, vitamin, energy drink, and prescribed foodstuff.

36. The foodstuff of any one of claim 1-26, wherein the foodstuff is a solid.

37. The foodstuff of any one of claim 1-26, wherein the foodstuff is a liquid.

38. The foodstuff of any one of claims 1-37, further comprising at least one additional therapeutic agent.

39. The foodstuff of claim 38, wherein the additional therapeutic agent is selected from the group consisting of an anti-cancer agent, an anti-inflammatory agent, an antiviral agent, an antibiotic agent, an antifungal agent, an antiparasitic agent, a bronchodilator, a vasopressor, a sedative, a complement inhibitor, an anti-coagulant, an immunomodulatory agent, an agent that induces tissue repair, an anticholinergic, an antidiarrheal, an antidepressant, a prokinetic agent, a laxative, a neurotransmitter, an antispasmodic, and a pain reliever.

40. The foodstuff of any one of claims 1-39 for the treatment of a disease or condition in a subject in need thereof.

41. The foodstuff of claim 40, wherein the disease or condition is associated with a low level of lαlp in a subject as compared to a reference level of lαlp.

42. The foodstuff of claim 40 or 41, wherein the disease or condition is associated with an altered level of at least one cytokine and/or chemokine in a subject as compared to a reference level of the at least one cytokine and/or chemokine.

43. The foodstuff of claim 42, wherein the cytokine and/or chemokine is TNF-α.

44. The foodstuff of any one of claims 40-43, wherein the disease or condition is selected from the group consisting of acute inflammatory disease, acute and chronic neurological and neurodegenerative disorders, sepsis, severe shock, septic shock, organ transplantation, organ failure, surgery, autoimmune disease, rheumatoid arthritis, multiple sclerosis, lupus, cancer, cancer metastasis, metabolic disorders, cachexia, trauma and/or injury, tissue damage, exposure to a toxin, liver disease, infectious disease, lung and respiratory disease, heart disease, kidney disease, ischemia, gastrointestinal disease, necrotizing enterocolitis, systemic inflammatory response syndrome (SIRS), rhinitis, exposure to a toxin, meningitis, acute pancreatitis, preeclampsia, preterm labor, primary immunodeficiency syndrome, and acquired immunodeficiency syndrome (AIDS).

45. The foodstuff of claim 44, wherein the inflammatory disease is an inflammatory bowel disease.

46. The foodstuff of claim 45, wherein the inflammatory bowel disease is Crohn's disease.

47. The foodstuff of claim 44, wherein the lung disease is an acute lung injury.

48. The foodstuff of claim 47, wherein the acute lung injury is acute respiratory distress syndrome (ARDS).

49. The foodstuff of claim 47, wherein the acute lung injury is pneumonia.

50. The foodstuff of claim 44, wherein the trauma and/or injury is a wound.

51. The foodstuff of claim 44, wherein the ischemia is ischemia/reperfusion injury.

52. The foodstuff of claim 44, wherein the ischemia is hypoxic ischemia.

53. The foodstuff of claim 44, wherein the ischemia is hypoxic ischemic encephalopathy.

54. The foodstuff of claim 44, wherein the disease or condition is necrotizing enterocolitis.

55. The foodstuff of claim 44, wherein the tissue damage is internal scarring, tissue damage resulting from organ transplantation or surgery, tissue damage resulting from inflammation, disease, or injury, lung tissue damage, brain tissue damage, gastrointestinal tissue damage, or vascular tissue damage.

56. A method of treating, reducing the symptoms of, inhibiting progression of, or reducing the likelihood of developing a disease or condition in a subject comprising administering to the subject a foodstuff comprising a therapeutically effective amount of an lαlp.

57. The method of claim 56, comprising administering to the subject in need thereof the foodstuff of any one of claims 1-39.

58. The method of claim 56 or 57, wherein the foodstuff is administered about every 4 to about 120 hours.

59. The method of any one of claims 56-58, wherein the foodstuff is administered at least once a day.

60. The method of claim 59, wherein the foodstuff is administered at least twice a day.

61. The method of any one of claims 56-60, wherein the foodstuff is administered over a treatment period.

62. The method of claim 61, wherein the treatment period is about 1 day to about 14 days.

63. The method of claim 61, wherein the treatment period is about 1 week to about 3 weeks.

64. The method of claim 61, wherein the treatment period is about 1 month to about 12 months.

65. The method of claim 61, wherein the treatment period is at least 1 year.

66. The method of any one of claims 56-65, further comprising determining the level of an lαlp in the subject.

67. The method of claim 66, wherein the level of the lαlp in the subject is determined prior to administration.

68. The method of claim 66 or 67, wherein the level of the lαlp in the subject is determined after administration.

69. The method of any one of claims 66-68, wherein the disease or condition is associated with a low level of lαlp in the subject as compared to a reference level of lαlp.

70. A method of determining whether a subject having a disease or condition is likely to respond to treatment with a foodstuff comprising lαlp comprising:
(a) optionally determining a pre-treatment level of one or more lαlps in the subject;
(b) administering a therapeutically effective amount of the foodstuff of any one of claims 1-39 to the subject; and
(c) determining the level of one or more of the lαlp in the subject after an initial treatment period, wherein an increase in the level of at least one of the lαlp in the subject indicates that the subject is likely to respond favorably to treatment with the foodstuff.

71. The method of claim 70, further comprising monitoring the level of one or more lαlp-related biomarkers in the subject prior to and/or post administration of the foodstuff comprising the lαlp.

72. A method of determining whether a subject having a disease or condition is likely to respond to treatment with a foodstuff comprising an lαlp comprising:
(a) optionally determining a pre-treatment level of one or more lαlp-related biomarkers in the subject;
(b) administering a therapeutically effective amount of the foodstuff of any one of claims 1-39 to the subject; and
(c) determining the level of one or more of the lαlp-related biomarkers in the subject after an initial treatment period, wherein a change in the level of at least one of the lαlp-related biomarkers in the subject indicates that the subject is likely to respond favorably to treatment with the foodstuff.

73. The method of claim 71 or 72, wherein the lαlp-related biomarker is selected from the group consisting of histone, extracellular histone, histone/Pαl complexes, histone/Ial complexes, histone lαl/Pαl complexes, TNF-α, IL-6, IL-10, IL-1, IL-1ra, IL1B, IL-8, MCP-1, MIP-2, C-reactive protein (CRP), procalcitonin (PCT), cytokine-induced neutrophil chemoattractant/KC, UTI, complement components C1, C2, C3, C4, C5, C6, C7, C8, C9, membrane attack complex, Factor B, Factor D, MASP-1, and MASP-2, or fragments thereof.

74. A method of optimizing therapeutic efficacy of a treatment of a subject having a disease or condition with a foodstuff comprising an lαlp, the method comprising:
(a) optionally determining a pre-treatment level of one or more lαlps in the subject;
(b) administering a therapeutically effective amount of the foodstuff of any one of claims 1-39 to the subject;
(c) determining the level of one or more of the lαlps in the subject after an initial treatment period, wherein (i) an increase in the level of at least one of the lαlps in the subject indicates that the foodstuff can be administered to the subject at a similar or reduced dosage or frequency, and (ii) a decrease or plateau in the level of at least one of the lαlps in the subject indicates that the foodstuff can be administered to the subject at an increased frequency or dosage; and
(d) optionally adjusting the frequency and/or the dosage at which the foodstuff is administered to the subject.

75. The method of any one of claims 56-74, wherein the disease or condition is associated with an elevated level of at least one cytokine and/or chemokine in the subject as compared to a reference level of the at least one cytokine and/or chemokine.

76. The method of claim 75, wherein the cytokine and/or chemokine is selected from the group consisting of IL-1β, TNF-α, INF-α, IL-6, IL-10, INF-γ, and IL-8.

77. The method of claim 75 or 76, wherein administration of the foodstuff results in a decrease in or down-regulation of one or more of the cytokines and/or chemokines.

78. The method of any one of claims 56-77, wherein the disease or condition is selected from the group consisting of acute inflammatory disease, acute and chronic neurological and neurodegenerative disorders, sepsis, severe shock, septic shock, organ transplantation, organ failure, surgery, autoimmune disease, rheumatoid arthritis, multiple sclerosis, lupus, cancer, cancer metastasis, metabolic disorders, cachexia, trauma and/or injury, tissue damage, exposure to a toxin, liver disease, infectious disease, lung and respiratory disease, heart disease, kidney disease, ischemia, gastrointestinal disease, necrotizing enterocolitis, systemic inflammatory response syndrome (SIRS), rhinitis, exposure to a toxin, meningitis, acute pancreatitis, preeclampsia, preterm labor, primary immunodeficiency syndrome, and acquired immunodeficiency syndrome (AIDS).

79. The method of claim 78, wherein the inflammatory disease is an inflammatory bowel disease.

80. The method of claim 79, wherein the inflammatory bowel disease is Crohn's disease.

81. The method of claim 78, wherein the lung disease is an acute lung injury.

82. The method of claim 81, wherein the acute lung injury is acute respiratory distress syndrome (ARDS).

83. The method of claim 81, wherein the acute lung injury is pneumonia.

84. The method of claim 78, wherein the trauma and/or injury is a wound.

85. The method of claim 78, wherein the ischemia is ischemia/reperfusion injury.

86. The method of claim 78, wherein the ischemia is hypoxic ischemia.

87. The method of claim 78, wherein the ischemia is hypoxic ischemic encephalopathy.

88. The method of claim 78, wherein the disease or condition is necrotizing enterocolitis.

89. The method of claim 78, wherein the tissue damage is internal scarring, tissue damage resulting from organ transplantation or surgery, tissue damage resulting from inflammation, disease, or injury, lung tissue damage, brain tissue damage, gastrointestinal tissue damage, or vascular tissue damage.

90. The method of any one of claims 56-89, wherein administration of the foodstuff reduces the frequency and/or occurrence of at least one symptom of the disease or condition in the subject, relative to an untreated subject.

91. The method of claim 90, wherein the symptom is selected from the group consisting of organ failure; hypoxemia; bilateral lung opacities; respiratory failure; dizziness, lightheadedness and/or fainting; fatigue; shortness of breath and/or labored breathing; cough; fever; abnormal vital signs, such as increased heart rate; low blood pressure; rapid breathing, chest pain and/or pressure; heart palpitations; edema; swelling, pain, and/or bloating of the abdomen; discoloration of the abdomen; pain in the lower joints and/or rectum; bloody stool; bowel obstruction; nausea; flatulence; loss of appetite; weight loss and/or poor weight gain; slow growth; diarrhea; poor feeding; vomiting; bleeding; redness, swelling, pain, tenderness and/or heat of the tissues proximal to a wound; blueish coloring of nails and/or lips; and the need for mechanical ventilation.

92. The method of any one of claims 56-91, wherein the foodstuff is administered at a dosage of about 1 mg/kg body weight to about 5 g/kg body weight of the subject.

93. The method of claim 92, wherein lαlp is present in the foodstuff in an amount of about 0.1 milligram (mg) to about 10 mg per mg of the foodstuff.

94. The method of claim 92, wherein the lαlp is present in the foodstuff in an amount of about 10 mg to about 1000 mg per liter (L) of the foodstuff.

95. The method of any one of claims 56-94, wherein the foodstuff is administered about every 4 to about 120 hours.

96. The method of claim 92 or 95, wherein the foodstuff is administered at least once a day.

97. The method of claim 96, wherein the foodstuff is administered at least twice a day.

98. The method of any one of claims 56-97, wherein the foodstuff is administered over a treatment period of at least 1 day.

99. The method of claim 98, wherein the treatment period is about 1 day to about 14 days.

100. The method of claim 98, wherein the treatment period is about 1 week to about 4 weeks.

101. The method of claim 98, wherein treatment period is about 1 month to about 12 months.

102. The method of claim 98, wherein the treatment period is at least 1 year.

103. The method of any one of claims 56-102, further comprising administering an additional therapeutic agent.

104. The method of claim 103, wherein the additional therapeutic agent is selected from the group consisting of an anti-cancer agent, an anti-inflammatory agent, an antiviral agent, an antibiotic agent, an antifungal agent, an antiparasitic agent, a bronchodilator, a vasopressor, a sedative, a complement inhibitor, an anti-coagulant, an immunomodulatory agent, an agent that induces tissue repair, an anticholinergic, an antidiarrheal, an antidepressant, a prokinetic agent, a laxative, a neurotransmitter, an antispasmodic, and a pain reliever.

105. The method of any one of claims 56-104, wherein the subject is a mammal.

106. The method of claim 105, wherein the subject is a human.

107. The method of claim 105 or 106, wherein the subject is a fetus, neonate, infant, child, adolescent, or adult.

108. A method of purifying an lαlp comprising: (a) separating a fraction of milk comprising the lαlp, and (b) purifying the lαlp from the fraction of milk, wherein the lαlp has with a purity ranging from about 85% to about 100%.

109. The method of claim 108, wherein the separating and/or purifying comprises a clarification step, a chromatography step, a precipitation step, and/or a solid phase extraction step.

110. The method of claim 109, wherein the chromatography step comprises anion-exchange and/or affinity chromatography.

111. The method of claim 109, wherein the precipitation step comprises contacting the fraction of milk with an agent that produces a precipitate lacking the lαlp.

112. The method of any one of claims 108-111, further comprising exposing the lαlp to a pH of about

5. 5 or lower, optionally a pH of about 4.2 to about 5.2.

113. The method of any one of claims 108-112, wherein fat and/or milk proteins are removed from the sample prior to chromatographic separation.

114. The method any one of claims 108-113, wherein the milk is from a mammal.

115. The method of claim 114, wherein the mammal is a domesticated ungulate.

116. The method of claim 115, wherein the domesticated ungulate is selected from the group consisting of a cow, goat, sheep, buffalo, camel, donkey, horse, reindeer, and yak.

117. The method of any one of claims 114-116, wherein the lαlp is expressed recombinantly in the mammal and secreted into the milk of the mammal.

118. The method of claim 117, wherein the lαlp is a human lαlp.

119. The method of any one of claims 108-118, wherein the lαlp has an apparent molecular weight of between about 60 kDa to about 280 kDa.

120. The method of claim 119, wherein the molecular weight is determined by sodium dodecyl sulfate polyacrylamide gel electrophoresis.

121. The method of any one of claims 108-120, wherein the lαlp has an in vivo half-life of greater than one hour.

122. The method of claim 121, wherein the in vivo half-life is greater than five hours.

123. The method of any one of claims 108-122, wherein the lαlp has a biological activity.

124. The method of claim 123, wherein the biological activity is selected from the group consisting of a cytokine inhibitor activity, increase of cytokine activity, chemokine inhibitor activity, protease inhibitor activity, chondroitin sulfate binding, glycosaminoglygan binding activity, hyaluronic acid binding activity, complement binding activity, histone binding activity, Arg-Gly-Asp (RGD) domain binding activity, coagulation factor binding activity, cellular repair activity, and extracellular matrix protein binding activity.

125. The method of any one of claims 108-124, wherein the lαlp has a high trypsin inhibitory specific activity.

126. The method of claim 125, wherein the trypsin inhibitory specific activity is between about 1000 IU/mg to about 2000 IU/mg.

127. A method of purifying an lαlp comprising:
(a) providing a mammal comprising a milk-producing cell transfected with a transgene that comprises:
(i) a nucleic acid sequence encoding the lαlp,
(ii) a milk-specific promoter, said promoter being operably linked to the nucleic acid sequence encoding the lαlp, and
(iii) a leader sequence encoding a protein secretory signal that enables secretion of the lαlp by the milk-producing cell; and
(b) purifying the lαlp from milk collected from the mammal.

128. The method of claim 127, wherein the lαlp is exogenous to the mammal.

129. The method of claim 127 or 128, wherein the mammal is a domesticated ungulate.

130. The method of claim 129, wherein the domesticated ungulate is selected from the group consisting of a cow, goat, sheep, buffalo, camel, donkey, horse, reindeer, and yak.

131. The method of any one of claims 128-130, wherein the lαlp is a human lαlp.

132. The method of any one of claims 127-131, wherein the lαlp is lαl, Pαl, H1, H2, H3, H4, H5, bikunin, or a combination thereof.

133. The method of claim 132, wherein the lαlp comprises lαl, Pαl, and/or bikunin.

134. The method of claim 132 or 133, wherein the lαlp comprises H1, H2, H3, H4, and/or H5.

135. The method of any one of claims 132-134, wherein the lαlp comprises bikunin.

136. A method of making a composition for oral consumption comprising admixing an lαlp with a foodstuff.

137. The method of claim 136, wherein the lαlp is lαl, Pαl, H1, H2, H3, H4, H5, bikunin, or a combination thereof.

138. The method of claim 137, wherein the lαlp comprises lαl, Pαl, and/or bikunin.

139. The method of claim 137 or 138, wherein the lαlp comprises H1, H2, H3, H4, and/or H5.

140. The method of any one of claims 136-139, wherein the lαlp comprises bikunin.

141. The method of any one of claims 136-140, wherein the lαlp admixed with the foodstuff ranges in purity from about 85% to about 100% pure.

142. The method of any one of claims 136-141, wherein the amount of lαlp admixed with the foodstuff ranges from about 0.1 milligram (mg) to about 10 mg per mg of the foodstuff.

143. The method of any one of claims 136-141, wherein the amount of lαlp admixed with the foodstuff ranges from about 10 mg to about 1000 mg per liter (L) of the foodstuff.

144. The method of any one of claims 136-143, wherein the lαlp is isolated from blood or milk.

145. The method of claim 144, wherein the blood or milk is from a mammal.

146. The method of claim 145, wherein the mammal is a domesticated ungulate.

147. The method of claim 146, wherein the domesticated ungulate is selected from the group consisting of a cow, goat, sheep, buffalo, camel, donkey, horse, reindeer, and yak.

148. The method of any one of claims 145-147, wherein the lαlp is expressed recombinantly in the mammal.

149. The method of claim 148, wherein the lαlp is a human lαlp.

150. The method of claim 145, wherein the lαlp is a human lαlp.

151. The method of any one of claims 136-150, wherein the lαlp has an apparent molecular weight of between about 60 kDa to about 280 kDa.

152. The method of claim 151, wherein the molecular weight is determined by sodium dodecyl sulfate polyacrylamide gel electrophoresis.

153. The method of any one of claims 136-152, wherein the lαlp has an in vivo half-life of greater than one hour.

154. The method of claim 153, wherein the in vivo half-life is greater than five hours.

155. The method of any one of claims 136-154, wherein the lαlp has a biological activity.

156. The method of claim 155, wherein the biological activity is selected from the group consisting of a cytokine inhibitor activity, increase of cytokine activity, chemokine inhibitor activity, protease inhibitor activity, chondroitin sulfate binding, glycosaminoglygan binding activity, hyaluronic acid binding activity, complement binding activity, histone binding activity, Arg-Gly-Asp (RGD) domain binding activity, coagulation factor binding activity, cellular repair activity, and extracellular matrix protein binding.

157. The method of any one of claims 136-156, wherein the lαlp has a high trypsin inhibitory specific activity.

158. The method of claim 157, wherein the trypsin inhibitory specific activity is between about 1000 IU/mg to about 2000 IU/mg.

159. The method of any one of claims 136-158, wherein the foodstuff is selected from the group consisting of a beverage, a milk-based product, a baked good, a fruit and/or vegetable-based product, a grain and/or cereal-based product, a non-dairy product, an infant formula, an electrolyte product, a sports drink, a protein-based product, a nutritional supplement, a food additive, a flavoring, a sweetener, a preservative, a food coloring agent, and a fiber.

160. The method of claim 159, wherein the milk-based product is selected from the group consisting of milk, cream, butter, yogurt, kefir, ice cream, gelato, sherbet, custard, pudding, nougat, cheese, a whey product, and a casein product.

161. The method of claim 159, wherein the baked good is selected from the group consisting of a biscuit, bread, brownie, cake, casserole, cookie, cracker, pastry, pie, pizza, and tart.

162. The method of claim 159, wherein the fruit and/or vegetable-based product is selected from the group consisting of an oil, jelly, jam, marmalade, preserve, butter, puree, infant food, sauce, soup, and broth.

163. The method of claim 159, wherein the non-dairy product is selected from the group consisting of a cheese substitute, non-dairy yogurt, non-dairy cream, non-dairy butter, non-dairy ice cream, non-dairy milk, tofu, soy-based product, nut-based product, coconut-based product, and gelatin.

164. The method of claim 159, wherein the cereal-based product is selected from the group consisting of bread, pasta, oatmeal, breakfast cereal, tortilla, and grits.

165. The method of claim 159, wherein the infant formula is selected from the group consisting of a protein hydrolysate formula, metabolic formula, amino acid based formula, exempt infant formula, specialized formula, follow-on formula, and a toddler formula.

166. The method of claim 159, wherein the electrolyte product is selected from the group consisting of a pre-mixed solution, a dissolvable tablet, an edible gel, a concentrated solution, and a powder.

167. The method of claim 159, wherein the electrolyte product and/or the sports drink is selected from the group consisting of an isotonic, hypertonic, and hypotonic solution.

168. The method of claim 159, wherein the nutritional supplement and/or protein-based product is selected from the group consisting of a meal replacement product, protein or nutritional shake, protein bar, vitamin, energy drink, and prescribed foodstuff.

169. The method of any one of claims 136-159, wherein the foodstuff is a solid.

170. The method of any one of claims 136-159, wherein the foodstuff is a liquid.

171. The method of any one of claims 136-170, wherein the lαlp is present in the foodstuff in an amount of about 0.1 mg to about 10 mg per mg of the foodstuff.

172. The method of any one of claims 136-170, wherein the lαlp is present in the foodstuff in an amount of about 10 mg to about 1000 mg per L of the foodstuff.

173. The method of any one of claims 136-172, wherein the lαlp comprises about 1% to about 60% of the volume of the foodstuff.

174. The method of any one of claims 136-173, further comprising admixing at least one additional therapeutic agent with the foodstuff.

175. The method of claim 174, wherein the additional therapeutic agent is selected from the group consisting of an anti-cancer agent, an anti-inflammatory agent, an antiviral agent, an antibiotic agent, an antifungal agent, an antiparasitic agent, a bronchodilator, a vasopressor, a sedative, a complement inhibitor, an anti-coagulant, an immunomodulatory agent, an agent that induces tissue repair, an anticholinergic, an antidiarrheal, an antidepressant, a prokinetic agent, a laxative, a neurotransmitter, an antispasmodic, and a pain reliever.

176. A kit comprising the foodstuff of any one of claims 1-39 and instructions for therapeutic use.

177. A kit comprising a composition comprising an lαlp, a foodstuff, instructions for admixing the composition with the foodstuff, and, optionally, instructions for therapeutic use.

178. The kit of claim 176 or 177, wherein the composition further comprises an additional therapeutic agent.

179. The kit of claim 178, wherein the additional therapeutic agent is selected from the group consisting of an anti-cancer agent, an anti-inflammatory agent, an antiviral agent, an antibiotic agent, an antifungal agent, an antiparasitic agent, a bronchodilator, a vasopressor, a sedative, a complement inhibitor, an anti-coagulant, an immunomodulatory agent, an agent that induces tissue repair, an anticholinergic, an antidiarrheal, an antidepressant, a prokinetic agent, a laxative, a neurotransmitter, an antispasmodic, and a pain reliever.

180. A method of treating, reducing the symptoms of, inhibiting progression of, or reducing the likelihood of developing necrotizing enterocolitis in a subject in need thereof comprising administering to the subject a composition comprising in admixture a therapeutically effective amount of an lαlp.

181. The method of claim 180, wherein the lαlp is lαl, Pαl, H1, H2, H3, H4, H5, bikunin, or a combination thereof.

182. The method of claim 181, wherein the lαlp comprises lαl, Pαl, and/or bikunin.

183. The method of claim 180 or 181, wherein the lαlp comprises H1, H2, H3, H4, and/or H5.

184. The method of any one of claims 180-183, wherein the lαlp comprises bikunin.

185. The method of any one of claims 180-184, wherein the lαlp ranges in purity from about 85% to about 100% pure.

186. The method of any one of claims 180-185, wherein the lαlp is isolated from blood or milk.

187. The method of claim 186, wherein the blood or milk is from a mammal.

188. The method of any one of claims 180-187, comprising administering the foodstuff of any one of claims 1-39.

189. The method of any one of claims 180-188, wherein the lαlp is administered about every 4 to about 120 hours.

190. The method of any one of claims 180-189, wherein the lαlp is administered at least once a day.

191. The method of claim 190, wherein the lαlp is administered at least twice a day.

192. The method of any one of claims 180-191, wherein the lαlp is administered over a treatment period.

193. The method of claim 192, wherein the treatment period is about 1 day to about 14 days.

194. The method of claim 192, wherein the treatment period is about 1 week to about 4 weeks.

195. The method of claim 192, wherein the treatment period is about 1 month to about 12 months.

196. The method of claim 192, wherein the treatment period is at least 1 year.

197. The method of any one of claims 180-196, further comprising determining the level of an lαlp and/or an lαlp-related biomarker in the subject.

198. The method of claim 197, wherein the lαlp-related biomarker is selected from the group consisting of histone, extracellular histone, histone/Pαl complexes, histone/lαl complexes, histone lαl/Pαl complexes, TNF-α, IL-6, IL-10, IL-1, IL-1ra, IL1B, IL-8, MCP-1, MIP-2, C-reactive protein (CRP), procalcitonin (PCT), cytokine-induced neutrophil chemoattractant/KC, UTI, complement components C1, C2, C3, C4, C5, C6, C7, C8, C9, membrane attack complex, Factor B, Factor D, MASP-1, and MASP-2, or fragments thereof.

199. The method of claim 197 or 198, wherein the level of the lαlp and/or an lαlp-related biomarker in the subject is determined prior to administration of the composition.

200. The method of any one of claims 197-199, wherein the level of the lαlp and/or lαlp-related biomarker in the subject is determined after administration of the composition.

201. The method of any one of claims 180-200, wherein the lαlp is administered at a dosage of about 1 mg/kg body weight to about 5 g/kg body weight.

202. The method of any one of claims 180-201, wherein the composition comprises a pharmaceutically acceptable excipient, diluent, or carrier.

203. The method of claim 202, wherein the composition is a solid.

204. The composition of claim 203, wherein said solid is a tablet, capsule, or suppository.

205. The method of claim 202, wherein the composition is a liquid.

206. The method of claim 202, wherein the composition is formulated for injection, infusion, inhalation, insufflation, or nebulization, or for oral, rectal, or topical administration.

207. The method of claim 206, wherein the injection is intravenous, intraperitoneal, or intracerebral injection.

208. The method of claim 207, wherein the infusion is fetal infusion.

209. The method of any one of claims 180-208, further comprising administering an additional therapeutic agent.

210. The method of claim 209, wherein the additional therapeutic agent is selected from the group consisting of anti-cancer agent, an anti-inflammatory agent, an antiviral agent, an antibiotic agent, an antifungal agent, an antiparasitic agent, a bronchodilator, a vasopressor, a sedative, a complement inhibitor, an anti-coagulant, an immunomodulatory agent, an agent that induces tissue repair, an anticholinergic, an antidiarrheal, an antidepressant, a prokinetic agent, a laxative, a neurotransmitter, an antispasmodic, and a pain reliever.

211. The method of any one of claims 180-210, wherein the subject is a mammal.

212. The method of claim 211, wherein the subject is a human.

213. The method of claim 211 or 212, wherein the subject is a fetus, neonate, infant, child, adolescent, or adult.

214. The method of any one of claims 180-213, comprising administering a therapeutically effective amount of the foodstuff of any one of claims 1-39 to the subject.

215. The method of any one of claims 180-214, wherein the lαlp is a human lαlp.

216. The foodstuff of claim 2, wherein the lαlp comprises H1, H2, H3, H4, and/or H5.

217. The foodstuff of claim 2, wherein the lαlp comprises bikunin.

218. The foodstuff of claim 1, wherein the lαlp admixed with the foodstuff ranges in purity from about 85% to about 100% pure.

219. The foodstuff of claim 1, wherein the lαlp is present in the foodstuff in an amount of about 0.1 milligram (mg) to about 10 mg per mg of the foodstuff.

220. The foodstuff of claim 1, wherein the lαlp is present in the foodstuff in an amount of about 10 mg to about 1000 mg per liter (L) of the foodstuff.

221. The foodstuff of claim 1, wherein the lαlp is isolated from blood or milk.

222. The foodstuff of claim 221, wherein the blood or milk is from a mammal.

223. The foodstuff of claim 222, wherein the mammal is a domesticated ungulate.

224. The foodstuff of claim 222, wherein the lαlp is expressed recombinantly in the mammal.

225. The foodstuff of claim 224, wherein the lαlp is a human lαlp.

226. The foodstuff of claim 1, wherein the foodstuff is pasteurized at a dry heat between about 50° C. and about 120° C.

227. The foodstuff of claim 1, wherein the foodstuff comprises at least one pharmaceutically acceptable excipient, diluent, carrier, and/or stabilizer.

228. The foodstuff of claim 227, wherein the stabilizer is selected from the group consisting of albumin, polyethylene glycol, alpha-trehalose, amino acids, salts, glycerol, omega-amino acids, sugar, and combination thereof.

229. The foodstuff of claim 1, wherein the foodstuff is selected from the group consisting of a beverage, a milk-based product, a baked good, a fruit and/or vegetable-based product, a grain and/or cereal-based product, a non-dairy product, an infant formula, an electrolyte product, a sports drink, a protein-based product, a nutritional supplement, a food additive, a flavoring, a sweetener, a preservative, a food coloring agent, and a fiber.

230. The foodstuff of claim 229, wherein the milk-based product is selected from the group consisting of milk, cream, butter, yogurt, kefir, ice cream, gelato, sherbet, custard, pudding, nougat, cheese, a whey product, and a casein product.

231. The foodstuff of claim 229, wherein the baked good is selected from the group consisting of a biscuit, bread, brownie, cake, casserole, cookie, cracker, pastry, pie, pizza, and tart.

232. The foodstuff of claim 229, wherein the fruit and/or vegetable-based product is selected from the group consisting of an oil, jelly, jam, marmalade, preserve, butter, puree, infant food, sauce, soup, and broth.

233. The foodstuff of claim 229, wherein the non-dairy product is selected from the group consisting of a cheese substitute, non-dairy yogurt, non-dairy cream, non-dairy butter, non-dairy ice cream, non-dairy milk, tofu, soy-based product, nut-based product, coconut-based product, and gelatin.

234. The foodstuff of claim 229, wherein the cereal-based product is selected from the group consisting of bread, pasta, oatmeal, breakfast cereal, tortilla, and grits.

235. The foodstuff of claim 229, wherein the infant formula is selected from the group consisting of a protein hydrolysate formula, metabolic formula, amino acid based formula, exempt infant formula, specialized formula, follow-on formula, and a toddler formula.

236. The foodstuff of claim 229, wherein the electrolyte product is selected from the group consisting of a pre-mixed solution, a dissolvable tablet, an edible gel, a concentrated solution, and a powder.

237. The foodstuff of claim 229, wherein the electrolyte product and/or the sports drink is selected from the group consisting of an isotonic, hypertonic, and hypotonic solution.

238. The foodstuff of claim 229, wherein the nutritional supplement and/or protein-based product is selected from the group consisting of a meal replacement product, protein or nutritional shake, protein bar, vitamin, energy drink, and prescribed foodstuff.

239. The foodstuff of claim 1, wherein the foodstuff is a solid.

240. The foodstuff of claim 1, wherein the foodstuff is a liquid.

241. The foodstuff of claim 1, further comprising at least one additional therapeutic agent.

242. The foodstuff of claim 241, wherein the additional therapeutic agent is selected from the group consisting of an anti-cancer agent, an anti-inflammatory agent, an antiviral agent, an antibiotic agent, an antifungal agent, an antiparasitic agent, a bronchodilator, a vasopressor, a sedative, a complement inhibitor, an anti-coagulant, an immunomodulatory agent, an agent that induces tissue repair, an anticholinergic, an antidiarrheal, an antidepressant, a prokinetic agent, a laxative, a neurotransmitter, an antispasmodic, and a pain reliever.

243. The foodstuff of claim 1 for the treatment of a disease or condition in a subject in need thereof.

244. The foodstuff of claim 243, wherein the disease or condition is associated with a low level of lαlp in a subject as compared to a reference level of lαlp.

245. The foodstuff of claim 243, wherein the disease or condition is associated with an altered level of at least one cytokine and/or chemokine in a subject as compared to a reference level of the at least one cytokine and/or chemokine.

246. The foodstuff of claim 245, wherein the cytokine and/or chemokine is TNF-α.

247. The foodstuff of claim 243, wherein the disease or condition is selected from the group consisting of acute inflammatory disease, acute and chronic neurological and neurodegenerative disorders, sepsis, severe shock, septic shock, organ transplantation, organ failure, surgery, autoimmune disease, rheumatoid arthritis, multiple sclerosis, lupus, cancer, cancer metastasis, metabolic disorders, cachexia, trauma and/or injury, tissue damage, exposure to a toxin, liver disease, infectious disease, lung and respiratory disease, heart disease, kidney disease, ischemia, gastrointestinal disease, necrotizing enterocolitis, systemic inflammatory response syndrome (SIRS), rhinitis, exposure to a toxin, meningitis, acute pancreatitis, preeclampsia, preterm labor, primary immunodeficiency syndrome, and acquired immunodeficiency syndrome (AIDS).

248. The foodstuff of claim 44, wherein the inflammatory disease is an inflammatory bowel disease.

249. The foodstuff of claim 45, wherein the inflammatory bowel disease is Crohn's disease.

250. The foodstuff of claim 44, wherein the disease or condition is tissue damage.

251. The method of claim 56, comprising administering to the subject in need thereof the foodstuff of claim 1.

252. The method of claim 56, wherein the foodstuff is administered about every 4 to about 120 hours.

253. The method of claim 56, wherein the foodstuff is administered at least once a day.

254. The method of claim 253, wherein the foodstuff is administered at least twice a day.

255. The method of claim 56, wherein the foodstuff is administered over a treatment period.

256. The method of claim 255, wherein the treatment period is about 1 day to about 14 days.

257. The method of claim 255, wherein the treatment period is about 1 week to about 3 weeks.

258. The method of claim 255, wherein the treatment period is about 1 month to about 12 months.

259. The method of claim 255, wherein the treatment period is at least 1 year.

260. The method of claim 56, further comprising determining the level of an lαlp in the subject.

261. The method of claim 260, wherein the level of the lαlp in the subject is determined prior to administration.

262. The method of claim 260, wherein the level of the lαlp in the subject is determined after administration.

263. The method of claim 260, wherein the disease or condition is associated with a low level of lαlp in the subject as compared to a reference level of lαlp.

264. The method of claim 56, wherein the disease or condition is associated with an elevated level of at least one cytokine and/or chemokine in the subject as compared to a reference level of the at least one cytokine and/or chemokine.

265. The method of claim 264, wherein the cytokine and/or chemokine is selected from the group consisting of IL-1βTNF-α, INF-α, IL-6, IL-10, INF-γ, and IL-8.

266. The method of claim 264, wherein administration of the foodstuff results in a decrease in or down-regulation of one or more of the cytokines and/or chemokines.

267. The method of claim 56, wherein the disease or condition is selected from the group consisting of acute inflammatory disease, acute and chronic neurological and neurodegenerative disorders, sepsis, severe shock, septic shock, organ transplantation, organ failure, surgery, autoimmune disease, rheumatoid arthritis, multiple sclerosis, lupus, cancer, cancer metastasis, metabolic disorders, cachexia, trauma and/or injury, tissue damage, exposure to a toxin, liver disease, infectious disease, lung and respiratory disease, heart disease, kidney disease, ischemia, gastrointestinal disease, necrotizing enterocolitis, systemic inflammatory response syndrome (SIRS), rhinitis, exposure to a toxin, meningitis, acute pancreatitis, preeclampsia, preterm labor, primary immunodeficiency syndrome, and acquired immunodeficiency syndrome (AIDS).

268. The method of claim 267, wherein the inflammatory disease is an inflammatory bowel disease.

269. The method of claim 268, wherein the inflammatory bowel disease is Crohn's disease.

270. The method of claim 267, wherein the disease or condition is tissue damage.

271. The method of claim 56, wherein administration of the foodstuff reduces the frequency and/or occurrence of at least one symptom of the disease or condition in the subject, relative to an untreated subject.

272. The method of claim 271, wherein the symptom is selected from the group consisting of organ failure; hypoxemia; bilateral lung opacities; respiratory failure; dizziness, lightheadedness and/or fainting; fatigue; shortness of breath and/or labored breathing; cough; fever; abnormal vital signs, such as increased heart rate; low blood pressure; rapid breathing, chest pain and/or pressure; heart palpitations; edema; swelling, pain, and/or bloating of the abdomen; discoloration of the abdomen; pain in the lower joints and/or rectum; bloody stool; bowel obstruction; nausea; flatulence; loss of appetite; weight loss and/or poor weight gain; slow growth; diarrhea; poor feeding; vomiting; bleeding; redness, swelling, pain, tenderness and/or heat of the tissues proximal to a wound; blueish coloring of nails and/or lips; and the need for mechanical ventilation.

273. The method of claim 56, wherein the foodstuff is administered at a dosage of about 1 mg/kg body weight to about 5 g/kg body weight of the subject.

274. The method of claim 273, wherein lαlp is present in the foodstuff in an amount of about 0.1 milligram (mg) to about 10 mg per mg of the foodstuff.

275. The method of claim 273, wherein the lαlp is present in the foodstuff in an amount of about 10 mg to about 1000 mg per liter (L) of the foodstuff.

276. The method of claim 56, wherein the foodstuff is administered about every 4 to about 120 hours.

277. The method of claim 273, wherein the foodstuff is administered at least once a day.

278. The method of claim 277, wherein the foodstuff is administered at least twice a day.

279. The method of claim 56, wherein the foodstuff is administered over a treatment period of at least 1 day.

280. The method of claim 279, wherein the treatment period is about 1 day to about 14 days.

281. The method of claim 279, wherein the treatment period is about 1 week to about 4 weeks.

282. The method of claim 279, wherein treatment period is about 1 month to about 12 months.

283. The method of claim 279, wherein the treatment period is at least 1 year.

284. The method of claim 56, further comprising administering an additional therapeutic agent.

285. The method of claim 284, wherein the additional therapeutic agent is selected from the group consisting of an anti-cancer agent, an anti-inflammatory agent, an antiviral agent, an antibiotic agent, an antifungal agent, an antiparasitic agent, a bronchodilator, a vasopressor, a sedative, a complement inhibitor, an anti-coagulant, an immunomodulatory agent, an agent that induces tissue repair, an anticholinergic, an antidiarrheal, an antidepressant, a prokinetic agent, a laxative, a neurotransmitter, an antispasmodic, and a pain reliever.

286. The method of claim 108, further comprising exposing the lαlp to a pH of about 5.5 or lower, optionally a pH of about 4.2 to about 5.2.

287. The method of claim 108, wherein fat and/or milk proteins are removed from the sample prior to chromatographic separation.

288. The method of claim 108, wherein the milk is from a mammal.

289. The method of claim 288, wherein the mammal is a domesticated ungulate.

290. The method of claim 288, wherein the lαlp is expressed recombinantly in the mammal and secreted into the milk of the mammal.

291. The method of claim 290, wherein the lαlp is a human lαlp.

292. The method of claim 127, wherein the mammal is a domesticated ungulate.

293. The method of claim 128, wherein the lαlp is a human lαlp.

294. The method of claim 127, wherein the lαlp is lαl, Pαl, H1, H2, H3, H4, H5, bikunin, or a combination thereof.

295. The method of claim 294, wherein the lαlp comprises lαl, Pαl, and/or bikunin.

296. The method of claim 294, wherein the lαlp comprises H1, H2, H3, H4, and/or H5.

297. The method of claim 294, wherein the lαlp comprises bikunin.

298. The method of claim 137, wherein the lαlp comprises H1, H2, H3, H4, and/or H5.

299. The method of claim 136, wherein the lαlp comprises bikunin.

300. The method of claim 136, wherein the lαlp admixed with the foodstuff ranges in purity from about 85% to about 100% pure.

301. The method of claim 136, wherein the amount of lαlp admixed with the foodstuff ranges from about 0.1 milligram (mg) to about 10 mg per mg of the foodstuff.

302. The method of claim 136, wherein the amount of lαlp admixed with the foodstuff ranges from about 10 mg to about 1000 mg per liter (L) of the foodstuff.

303. The method of claim 136, wherein the lαlp is isolated from blood or milk.

304. The method of claim 303, wherein the blood or milk is from a mammal.

305. The method of claim 304, wherein the mammal is a domesticated ungulate.

306. The method of claim 304, wherein the lαlp is expressed recombinantly in the mammal.

307. The method of claim 306, wherein the lαlp is a human lαlp.

308. The method of claim 304, wherein the lαlp is a human lαlp.

309. The method of claim 136, wherein the foodstuff is selected from the group consisting of a beverage, a milk-based product, a baked good, a fruit and/or vegetable-based product, a grain and/or cereal-based product, a non-dairy product, an infant formula, an electrolyte product, a sports drink, a protein-based product, a nutritional supplement, a food additive, a flavoring, a sweetener, a preservative, a food coloring agent, and a fiber.

310. The method of claim 309, wherein the milk-based product is selected from the group consisting of milk, cream, butter, yogurt, kefir, ice cream, gelato, sherbet, custard, pudding, nougat, cheese, a whey product, and a casein product.

311. The method of claim 309, wherein the baked good is selected from the group consisting of a biscuit, bread, brownie, cake, casserole, cookie, cracker, pastry, pie, pizza, and tart.

312. The method of claim 309, wherein the fruit and/or vegetable-based product is selected from the group consisting of an oil, jelly, jam, marmalade, preserve, butter, puree, infant food, sauce, soup, and broth.

313. The method of claim 309, wherein the non-dairy product is selected from the group consisting of a cheese substitute, non-dairy yogurt, non-dairy cream, non-dairy butter, non-dairy ice cream, non-dairy milk, tofu, soy-based product, nut-based product, coconut-based product, and gelatin.

314. The method of claim 309, wherein the cereal-based product is selected from the group consisting of bread, pasta, oatmeal, breakfast cereal, tortilla, and grits.

315. The method of claim 309, wherein the infant formula is selected from the group consisting of a protein hydrolysate formula, metabolic formula, amino acid based formula, exempt infant formula, specialized formula, follow-on formula, and a toddler formula.

316. The method of claim 309, wherein the electrolyte product is selected from the group consisting of a pre-mixed solution, a dissolvable tablet, an edible gel, a concentrated solution, and a powder.

317. The method of claim 309, wherein the electrolyte product and/or the sports drink is selected from the group consisting of an isotonic, hypertonic, and hypotonic solution.

318. The method of claim 309, wherein the nutritional supplement and/or protein-based product is selected from the group consisting of a meal replacement product, protein or nutritional shake, protein bar, vitamin, energy drink, and prescribed foodstuff.

319. The method of claim 136, wherein the foodstuff is a solid.

320. The method of claim 136, wherein the foodstuff is a liquid.

321. The method of claim 136, wherein the lαlp is present in the foodstuff in an amount of about 0.1 mg to about 10 mg per mg of the foodstuff.

322. The method of claim 136, wherein the lαlp is present in the foodstuff in an amount of about 10 mg to about 1000 mg per L of the foodstuff.

323. The method of claim 136, wherein the lαlp comprises about 1% to about 60% of the volume of the foodstuff.

324. The method of claim 136, further comprising admixing at least one additional therapeutic agent with the foodstuff.

325. The method of claim 324, wherein the additional therapeutic agent is selected from the group consisting of an anti-cancer agent, an anti-inflammatory agent, an antiviral agent, an antibiotic agent, an antifungal agent, an antiparasitic agent, a bronchodilator, a vasopressor, a sedative, a complement inhibitor, an anti-coagulant, an immunomodulatory agent, an agent that induces tissue repair, an anticholinergic, an antidiarrheal, an antidepressant, a prokinetic agent, a laxative, a neurotransmitter, an antispasmodic, and a pain reliever.

326. The method of claim 180, wherein the lαlp comprises H1, H2, H3, H4, and/or H5.

327. The method of claim 180, wherein the lαlp comprises bikunin.

328. The method of claim 180, wherein the lαlp ranges in purity from about 85% to about 100% pure.

329. The method of claim 180, wherein the lαlp is isolated from blood or milk.

330. The method of claim 329, wherein the blood or milk is from a mammal.

331. The method of claim 180, comprising administering the foodstuff of claim 1.

332. The method of claim 180, wherein the lαlp is administered about every 4 to about 120 hours.

333. The method of claim 180, wherein the lαlp is administered at least once a day.

334. The method of claim 333, wherein the lαlp is administered at least twice a day.

335. The method of claim 180, wherein the lαlp is administered over a treatment period.

336. The method of claim 335, wherein the treatment period is about 1 day to about 14 days.

337. The method of claim 335, wherein the treatment period is about 1 week to about 4 weeks.

338. The method of claim 335, wherein the treatment period is about 1 month to about 12 months.

339. The method of claim 335, wherein the treatment period is at least 1 year.

340. The method of claim 180, further comprising determining the level of an lαlp and/or an lαlp-related biomarker in the subject.

341. The method of claim 340, wherein the lαlp-related biomarker is selected from the group consisting of histone, extracellular histone, histone/Pαl complexes, histone/Ial complexes, histone lαl/Pαl complexes, TNF-α, IL-6, IL-10, IL-1, IL-1ra, IL1B, IL-8, MCP-1, MIP-2, C-reactive protein (CRP), procalcitonin (PCT), cytokine-induced neutrophil chemoattractant/KC, UTI, complement components C1, C2, C3, C4, C5, C6, C7, C8, C9, membrane attack complex, Factor B, Factor D, MASP-1, and MASP-2, or fragments thereof.

342. The method of claim 340, wherein the level of the lαlp and/or an lαlp-related biomarker in the subject is determined prior to administration of the composition.

343. The method of claim 340, wherein the level of the lαlp and/or lαlp-related biomarker in the subject is determined after administration of the composition.

344. The method of claim 180, wherein the lαlp is administered at a dosage of about 1 mg/kg body weight to about 5 g/kg body weight.

345. The method of claim 180, wherein the composition comprises a pharmaceutically acceptable excipient, diluent, or carrier.

346. The method of claim 345, wherein the composition is a solid.

347. The composition of claim 346, wherein said solid is a tablet, capsule, or suppository.

348. The method of claim 345, wherein the composition is a liquid.

349. The method of claim 345, wherein the composition is formulated for injection, infusion, inhalation, insufflation, or nebulization, or for oral, rectal, or topical administration.

350. The method of claim 349, wherein the injection is intravenous, intraperitoneal, or intracerebral injection.

351. The method of claim 350, wherein the infusion is fetal infusion.

352. The method of claim 180, further comprising administering an additional therapeutic agent.

353. The method of claim 352, wherein the additional therapeutic agent is selected from the group consisting of anti-cancer agent, an anti-inflammatory agent, an antiviral agent, an antibiotic agent, an antifungal agent, an antiparasitic agent, a bronchodilator, a vasopressor, a sedative, a complement inhibitor, an anti-coagulant, an immunomodulatory agent, an agent that induces tissue repair, an anticholinergic, an antidiarrheal, an antidepressant, a prokinetic agent, a laxative, a neurotransmitter, an antispasmodic, and a pain reliever.

354. The method of claim 180, wherein the subject is a mammal.

355. The method of claim 354, wherein the subject is a human.

356. The method of claim 354, wherein the subject is a fetus, neonate, infant, child, adolescent, or adult.

357. The method of claim 180, wherein the lαlp is a human lαlp.
